(19) United States
(12) Patent Application Publication
(10) Pub. No.: US 2009/0270452 A1
(43) Pub. Date: Oct. 29, 2009

(54) USE OF SOLUBLE EPOXIDE HYDROLASE INHIBITORS IN THE TREATMENT OF SMOOTH MUSCLE DISORDERS

(75) Inventors: Heather K. Webb Hsu, Seattle, WA (US); Yi-Xin Wang, Lafayette, CA (US); Le-Ning Zhang, Pleasant Hill, CA (US)

Correspondence Address: FOLEY & LARDNER LLP, 975 PAGE MILL ROAD, PALO ALTO, CA, 94304, US

(73) Assignee: Arete Therapeutics, Inc.

(21) Appl. No.: 12/426,154

(22) Filed: Apr. 17, 2009

Related U.S. Application Data
(60) Provisional application No. 61/046,156, filed on Apr. 18, 2008.
(60) Provisional application No. 61/056,776, filed on May 28, 2008.

(51) Int. Cl. A61K 31/4545; A61K 31/4468; A61K 31/17; A61P 21/00

(52) U.S. Cl. 514/318; 514/329; 514/588

(57) ABSTRACT

Disclosed herein are compounds, compositions, and methods for enhancing smooth muscle function in a subject by administration of soluble epoxide hydrolase inhibitors and for treating subjects with smooth muscle disorders including erectile dysfunction, overactive bladder, uterine contractions and irritable bowel syndrome.

### CROSS-REFERENCE TO RELATED APPLICATION

This application claims the benefit under 35 U.S.C. § 119(e) of U.S. Provisional Patent Application Ser. Nos. 61/046,156, filed on Apr. 18, 2008, and 61/056,776, filed on May 28, 2008, both of which are incorporated herein by reference in their entirety.

### FIELD OF THE INVENTION

The present invention generally relates to compounds and methods useful for improving smooth muscle function and for treating disorders associated therewith.

### BACKGROUND

The arachidonate cascade is a ubiquitous lipid signaling cascade that liberates arachidonic acid from the plasma membrane lipid reserves in response to a variety of extra-cellular and/or intra-cellular signals. The released arachidonic acid is then available to act as a substrate for a variety of oxidative enzymes that convert it to signaling lipids that have been implicated in inflammation and other diseases. Several commercially available drugs target and disrupt this pathway. Non-steroidal anti-inflammatory drugs (NSAIDs) disrupt the conversion of arachidonic acid to prostaglandins by inhibiting cyclooxygenases (COX1 and COX2). Asthma drugs, such as SINGULAIR™ disrupt the conversion of arachidonic acid to leukotrienes by inhibiting lipoxygenase (LOX).

Certain cytochrome P450-dependent enzymes convert arachidonic acid into a series of epoxide derivatives known as epoxyeicosatrienoic acids (EETs). These EETs are particularly prevalent in endothelium (cells that make up arteries and vascular beds), kidney, and lung. In contrast to many of the end products of the prostaglandin and leukotriene pathways, the EETs are reported to have a variety of anti-inflammatory and anti-hypertensive properties.

While EETs have potent effects in vivo, the epoxide moiety of the EETs is rapidly hydrolyzed into the less active dihydroxyeicosatrienoic acid (DHET) form by an enzyme called soluble epoxide hydrolase (sEH). Inhibition of sEH has been reported to significantly reduce blood pressure in hypertensive animals (see, e.g., Yu et al. Circ. Res. 87:992-8 (2000) and Sinal et al. J. Biol. Chem. 275:40504-10 (2000)), to reduce the production of proinflammatory nitric oxide (NO), cytokines, and lipid mediators, and to contribute to inflammatory resolution by enhancing lipoxin A₄ production in vivo (see Schmelzer et al. Proc. Nat'l Acad. Sci. USA 102(28):9772-7 (2005)).

In addition to the effects of EETs on vascular endothelial cells, EETs have been shown to be produced in rat penile endothelial cells and are implicated in the formation of a normal penile erection (Jin et al. FASEB J. 20:539-541 (2006)). Recently, the administration of EETs were examined for their ability to relax the corporal smooth muscle of rats (Yousif and Benter Vascul. Pharmacol. 47(5-6):281-287 (2007)). EETs were found to cause dose dependent relaxation of normal, old and diabetic rat corpus cavernosual strips with an apparent EC₅₀ of 10 nM and Eₘₐₓ of 30-70% depending on the system. Furthermore, phenylephrine (PE) induced contraction of the cavernosual strips was attenuated by preincubation with an inhibitor of 20-HETE synthesis (N′-(4-dutyl-2-methyl-phenyl)-foramidine) or an inhibitor of soluble epoxide hydrolase (1-cyclodexy-3-dodecyl-urea).

### SUMMARY OF THE INVENTION

This invention provides soluble epoxide hydrolase (sEH) inhibitor compounds and compositions thereof that are useful for improving smooth muscle function and in treating disorders associated therewith, including erectile dysfunction, overactive bladder, uterine contractions and irritable bowel syndrome.

In one aspect, the invention provides a method for enhancing non-vascular smooth muscle relaxation in a subject by administering to the subject an effective amount of a sEH inhibitor. In a further aspect the enhancement is unrelated to hypertension. In yet a further aspect, the non-vascular smooth muscle comprises the muscles of the reproductive organs, bladder, or gastrointestinal tract.

Another aspect provides a method for treating or preventing one or more non-vascular smooth muscle disorders in a subject, wherein the smooth muscle disorder is characterized by an otherwise healthy smooth muscle which over or under responds to stimuli and is not hypertension. These non-vascular smooth muscle disorders include, but are not limited to, erectile dysfunction, overactive bladder, uterine contractions or irritable bowel syndrome. This method comprises administering to the subject an amount of a sEH inhibitor effective to treat the disorder manifested in the subject. This method is particularly suited for a subject suffering from a non-vascular smooth muscle disorder who is unable to be treated with an effective amount of a phosphodiesterase type 5 inhibitor or an anticholinergic. In yet a further aspect, the subject further suffers from one or more conditions including congestive heart failure, heart disease, stroke, hypotension and diabetes.

The methods described herein preferably includes the administration of an effective amount of a sEH inhibitor, including but not limited to compounds of any one of Formulas (A), (I), to (V), or pharmaceutically acceptable salts thereof.

Accordingly, one class provided herein are sEH inhibitors of Formula (A) or a pharmaceutically acceptable salt thereof:

R¹-L-C(=Q)NR²R^{2a} (A)

wherein:
L is selected from the group consisting of —NH—, —CR^{a}R^{b}—, a covalent bond, and —CR^{a}R^{b}NH—, where R^{d} and R^{b} are independently hydrogen or alkyl, or R^{a} and R^{b} together with the carbon bound thereto form a C₃-C₆ cycloalkyl;
Q is selected from the group consisting of O and S; and
R¹ and R² are independently selected from the group consisting of substituted alkyl, aryl, substituted aryl, heteroaryl, substituted heteroaryl, cycloalkyl, substituted cycloalkyl, heterocycloalkyl, and substituted heterocycloalkyl;
R^{2a} is selected from the group consisting of hydrogen and alkyl;
or R² and R^{2a} together with the nitrogen bound thereto form an optionally substituted heterocycloalkyl;
provided that when L is not —NH—, R^{2a} is hydrogen.

In some embodiments, the compound is a member of the group of Formula (I) or a stereoisomer, tautomer, or pharmaceutically acceptable salt thereof:

R¹NHC(═O)NHR² (I)

wherein:
Q is selected from the group consisting of O and S; and
R¹ and R² are independently selected from the group consisting of substituted alkyl, aryl, substituted aryl, heteroaryl, substituted heteroaryl, cycloalkyl, substituted cycloalkyl, heterocycloalkyl, and substituted heterocycloalkyl.

In some embodiments, the compound is a member of the group of Formula (II) or (III):

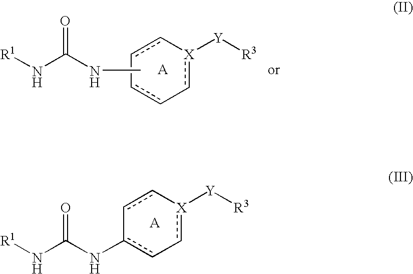

wherein:
R¹ is selected from the group consisting of aryl, substituted aryl, heteroaryl, substituted heteroaryl, cycloalkyl, substituted cycloalkyl, heterocycloalkyl, and substituted heterocycloalkyl;
X is CH, C or N; provided that when X is CH then ring A is cyclohexyl, when X is C then ring A is phenyl, and when X is N then ring A is piperidinyl;
Y is selected from the group consisting of CO, a covalent bond, O, and SO₂; and
R³ is selected from the group consisting of alkyl, substituted alkyl, heteroaryl, substituted heteroaryl, heterocycloalkyl and substituted heterocycloalkyl; or
a stereoisomer, tautomer, or pharmaceutically acceptable salt thereof.

Also provided are sEH inhibitors of Formula (IV):

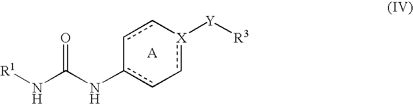

wherein:
R¹ is selected from the group consisting of aryl, substituted aryl, heteroaryl, substituted heteroaryl, cycloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂; and
R³ is selected from the group consisting of alkyl, substituted alkyl, heteroaryl, substituted heteroaryl, heterocycloalkyl and substituted heterocycloalkyl; or
a stereoisomer, tautomer, or pharmaceutically acceptable salt thereof.

Also provided are sEH inhibitors of Formula (V):

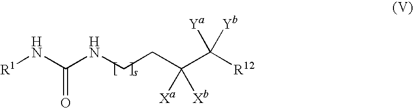

wherein:
R¹ is selected from the group consisting of aryl, substituted aryl, heteroaryl, substituted heteroaryl, cycloalkyl, substituted cycloalkyl, heterocycloalkyl, and substituted heterocycloalkyl;
s is 0-10;
R¹² is selected from the group consisting of —OR¹², —CH₂OR¹³, —COR¹³, —COOR¹³, —CONR¹³R¹⁴, and carboxylic acid isostere; and
R¹³ and R¹⁴ are independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; or R¹³ and R¹⁴ together with the nitrogen atom bound thereto form a heterocycloalkyl ring having 3 to 9 ring atoms, and wherein said ring is optionally substituted with alkyl, substituted alkyl, heterocycloalkyl, oxo or carboxy; and
each of X^{a}, X^{b}, Y^{a}, and Y^{b} is independently selected from the group consisting of hydrogen, C₁-C₄ alkyl, substituted C₁-C₄ alkyl and halo, provided that at least one of Y^{a} and Y^{b} is halo or C₁-C₄ alkyl; or
a stereoisomer, tautomer, or pharmaceutically acceptable salt thereof.

In a particular aspect of this invention, the compound to be administered is selected from the group consisting of:
1-adamantyl-3-(1-(methylsulfonyl)piperidin-4-yl)urea;
1-(1-nicotinoylpiperidin-4-yl)-3-(4-(trifluoromethoxy)phenyl)urea;
1-adamantyl-3-(1-acetylpiperidin-4-yl)urea;
ethyl 2-fluoro-8-(3-adamantylureido)octanoate;
2-fluoro-8-(3-adamantylureido)octanoic acid;
1-cyclohexyl-3-(1-picolinoylpiperidin-4-yl)urea;
1-(1-(isopropylsulfonyl)piperidin-4-yl)-3-(4-(trifluoromethyl)phenyl)urea;
4-(4-3-adamantan-1-yl-ureido)cyclohexyloxy)benzoic acid;
isopropyl 4-(3-(4-(trifluoromethoxy)phenyl)ureido)piperidine-1-carboxylate;
1-(1-acetyl-piperidin-4-yl)-3-(3-trifluoromethyl-phenyl)-urea;
1-(1-(isopropylsulfonyl)piperidin-4-yl)-3-(4-(trifluoromethoxy)phenyl)urea;
1-(4-methanesulfonyl-phenyl)-3-(4-trifluoromethyl-phenyl)-urea;
1-(1-(3,3-dimethylbutanoyl)piperidin-4-yl)-3-(4-(trifluoromethyl)phenyl)urea;
1-(1-acetyl-piperidin-4-yl)-3-(4-trifluoromethyl-phenyl)-urea; and
1-(1-methanesulfonyl-piperidin-4-yl)-3-(4-trifluoromethyl-phenyl)-urea.

In another aspect of this invention, provided is a method of treating a vascular smooth muscle disorder in a patient in need thereof, comprising administering a therapeutically effective amount of a compound selected from the group consisting of:
1-adamantyl-3-(1-(methylsulfonyl)piperidin-4-yl)urea;
1-(1-nicotinoylpiperidin-4-yl)-3-(4-(trifluoromethoxy)phenyl)urea; and
1-adamantyl-3-(1-acetylpiperidin-4-yl)urea.

### DESCRIPTION OF THE FIGURES

FIG. 1 shows two independent experiments (white bars represent experiment 1, whereas black bars represent experiment 2), wherein 1-adamantyl-3-(1-(methylsulfonyl)piperidin-4-yl)urea (Compound 1) and/or 14,15-EET were evaluated for their ability to relax normal rabbit corpus cavernosum strips. Exposure of the corpus cavernosum strips to both Compound 1 and 14,15-EET shows a dose dependent synergistic effect on relaxation of the strips. The X-axis represents the compound used and their 1M concentrations. The Y-axis represents the percent relaxation of the corpus cavernosum strips normalized to the vehicle control of corpus cavernosum strips incubated with sodium nitroprusside.

FIG. 2 shows direct vasorelaxation of rat mesenteric arteries with intact (open circles) or denuded (filled circles) endothelium incubated with 1-(1-nicotinoylpiperidin-4-yl)-3-(4-(trifluoromethoxy)phenyl)urea (Compound 2). The X-axis represents the 1M concentration of Compound 2. The Y-axis represents the percent relaxation normalized to pre-contraction length of the arteries. The abbreviation n equals the number of animals tested.

FIG. 3 shows direct vasorelaxation of rat mesenteric arteries pre-contracted by U46619, 9,11-dideoxy-9α,11α-methanoepoxy Prostaglandin F_{2α}, a thromboxane A2 agonist, (open circles) or high concentration of KCl (127 mM, filled circles) with 1-(1-nicotinoylpiperidin-4-yl)-3-(4-(trifluoromethoxy)phenyl)urea (Compound 2). The X-axis represents the μM concentration of Compound 2. The Y-axis represents the percent relaxation normalized to pre-contraction length of the arteries. The abbreviation n equals the number of animals tested.

FIG. 3A shows direct vasorelaxation response to EET (14,15-epoxyeicosatrienoic acid) in rat mesenteric arteries pre-contracted with U46619 (filed circles) or high KCl (100 mM, open circles). The X-axis represents the μM concentration of EET. The Y-axis represents the percent relaxation normalized to pre-contraction length of the arteries. The abbreviation n equals the number of animals tested.

FIG. 4 shows enhanced response to acetylcholine (Ach) induced relaxation of rat vascular smooth muscle by in vitro treatment with 1-(1-nicotinoylpiperidin-4-yl)-3-(4-(trifluoromethoxy)phenyl)urea (Compound 2, circles) or 1-adamantyl-3-(1-acetylpiperidin-4-yl)urea (Compound 3, squares). Open squares and circles represent muscle response to Ach before treatment with either compound. Filled squares and circles represent muscle response to Ach after treatment with either compound. The X-axis represents the nM concentration of acetylcholine. The Y-axis represents the percent relaxation normalized to pre-contraction length vascular smooth muscle.

FIG. 5 shows enhanced response to acetylcholine induced relaxation of rat vascular smooth muscle from rats chronically infused with angiotensin II (Ang II) by in vivo co-treatment with 1-(1-nicotinoylpiperidin-4-yl)-3-(4-(trifluoromethoxy)phenyl)urea (Compound 2, 10 mg/kg once a day by oral gavage are squares) or 1-adamantyl-3-(1-acetylpiperidin-4-yl)urea (Compound 3, ˜30 mg/kg dose per day in drinking water are triangles). Normal untreated control rats are represented by open circles, whereas positive control rats treated with angiotensin II (Ang II) are represented by filled circles. The X-axis represents the nM concentration of acetylcholine. The Y-axis represents the percent relaxation normalized to pre-contraction length vascular smooth muscle. The abbreviation n equals the number of animals tested.

FIG. 6 shows enhanced response to acetylcholine induced relaxation of vascular smooth muscle isolated from spontaneously hypertensive rats (SHR) by in vitro treatment with 1-adamantyl-3-(1-acetylpiperidin-4-yl)urea (Compound 3). The vehicle controls, represented by the filed squares, are untreated vascular smooth muscles. The filed circles are vascular smooth muscles treated with Compound 3. The X-axis represents the nM concentration of acetylcholine. The Y-axis represents the percent relaxation normalized to pre-contraction length vascular smooth muscle. The abbreviation n equals the number of animals tested.

FIG. 7 shows enhanced response to sodium nitroprusside (SNP) induced relaxation of vascular smooth muscle isolated from spontaneously hypertensive rats (SHR) by in vitro treatment with 1-adamantyl-3-(1-acetylpiperidin-4-yl)urea (Compound 3). The vehicle controls, represented by the filed squares, are untreated vascular smooth muscles. The filed circles are vascular smooth muscles treated with Compound 3. The X-axis represents the nM concentration of SNP. The Y-axis represents the percent relaxation normalized to pre-contraction length vascular smooth muscle. The abbreviation n equals the number of animals tested.

### DETAILED DESCRIPTION

Throughout this disclosure, various publications, patents and published patent specifications are referenced by an identifying citation. The disclosures of these publications, patents and published patent specifications are hereby incorporated by reference in their entirety into the present disclosure to more fully describe the state of the art to which this invention pertains.

As used herein, certain terms have the following defined meanings.

As used in the specification and claims, the singular form “a”, “an” and “the” include plural references unless the context clearly dictates otherwise. For example, the term “a pharmaceutically acceptable salt” includes a plurality of pharmaceutically acceptable salts, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace amount of elements of other ingredients and substantial method steps. Embodiments defined by each of these transition terms are within the scope of this invention.

“Cis-Epoxyeicosatrienoic acids” (“EETs”) are biomediators synthesized by cytochrome P450 epoxygenases.

“Epoxide hydrolases” (“EH;” EC 3.3.2.3) are enzymes in the alpha/beta hydrolase fold family that add water to 3 membered cyclic ethers termed epoxides.

“Soluble epoxide hydrolase” (“sEH”) is an enzyme which in endothelial, smooth muscle and other cell types converts EETs to dihydroxy derivatives called dihydroxyeicosatrienoic acids (“DHETs”). The term “Soluble epoxide hydrolase” (“sEH”) as used herein includes all functional genetic variants. The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)).

“sEH inhibitor” refers to an inhibitor that inhibits by 50% the activity of sEH in hydrolyzing epoxides at a concentration of less than about 500 μM, preferably, the inhibitor inhibits by 50% the activity of sEH in hydrolyzing epoxides at a concentration of less than about 100 μM, even more preferably, the inhibitor inhibits by 50% the activity of sEH in hydrolyzing epoxides at a concentration of less than about 100 nM, and most preferably, the inhibitor inhibits by 50% the activity of sEH in hydrolyzing epoxides at a concentration of less than about 50 nM. sEH inhibitors are further described herein. Preferably the sEH inhibitors for use in the methods of this invention are pharmaceutically acceptable compounds.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkenyl” refers to straight or branched hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR³¹R³² where R³¹ and R³² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R³¹ and R³² are optionally joined, together with the nitrogen bound thereto to form a heterocyclic or substituted heterocyclic group, provided that R³¹ and R³² are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R³¹ is hydrogen and R³² is alkyl, the substituted amino group is sometimes referred to herein as alkylamino. When R³¹ and R³² are alkyl, the substituted amino group is sometimes referred to herein as dialkylamino. When referring to a monosubstituted amino, it is meant that either R³¹ or R³² is hydrogen but not both. When referring to a disubstituted amino, it is meant that neither R³¹ nor R³² are hydrogen.

“Aminocarbonyl” refers to the group —C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²⁰C(O)NR¹⁰R¹¹ where R²⁰ is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NR²⁰C(S)NR¹⁰R¹¹ where R²⁰ is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR²⁰—SO₂NR¹⁰R¹¹ where R²⁰ is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR³²)NR¹⁰R¹¹ where R¹⁰, R¹¹, and R³² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein. “Substituted phenyl” refers to a phenyl group substituted with 1 to 5, preferably 1 to 3, or more preferably 1 to 2 substituents selected from the groups listed above.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)— which is equivalent to —C(═O)—.

“Carboxy” or “carboxyl” refers to —COOH or salts thereof.

“Isosteres” are different compounds that have different molecular formulae but exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated by the present invention include —SO₃H, —SO₂NHR^{j}, —PO₂(R^{j})₂, —CN, —PO₃(R^{j})₂, —OR^{j}, —SR^{j}, —NHCOR^{j}, —N(R^{j})₂, —CONH(O)R^{j}, —CONHNHSO₂R^{j}, —COHNSO₂R^{j}, —SO₂NHCOR^{j}, —SO₂NHNHCOR^{j}, and —CONR^{j}CN, where R^{j} is selected from hydrogen, hydroxyl, halo, haloalkyl, thiocarbonyl, alkoxy, alkenoxy, aryloxy, cyano, nitro, imino, alkylamino, aminoalkyl, thiol, thioalkyl, alkylthio, sulfonyl, alkyl, alkenyl, alkynyl, aryl, aralkyl(-(alkyl)-(aryl)), cycloalkyl, heteroaryl, heterocycle, and CO₂R^{m} where R^{m} is hydrogen, alkyl or alkenyl. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of preferred carboxylic acid isosteres contemplated by this invention.

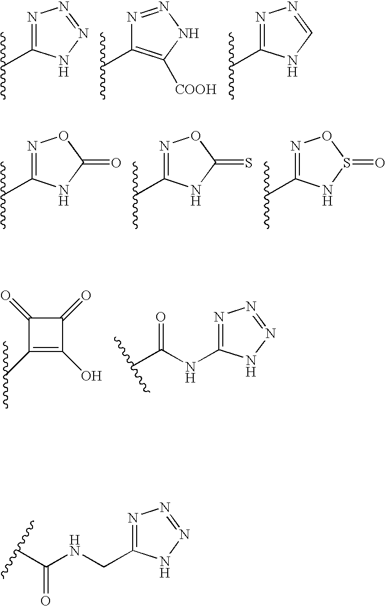

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR²⁰—C(O)O-alkyl, —NR²⁰—C(O)O-substituted alkyl, —NR²⁰—C(O)O-alkenyl, —NR²⁰—C(O)O-substituted alkenyl, —NR²⁰—C(O)O-alkynyl, —NR²⁰—C(O)O-substituted alkynyl, —NR²⁰—C(O)O-aryl, —NR²⁰—C(O)O-substituted aryl, —NR²⁰—C(O)O-cycloalkyl, —NR²⁰—C(O)O-substituted cycloalkyl, —NR²⁰—C(O)O-cycloalkenyl, —NR²⁰—C(O)O-substituted cycloalkenyl, —NR²⁰—C(O)O-heteroaryl, —NR²⁰—C(O)O-substituted heteroaryl, —NR²⁰—C(O)O-heterocyclic, and —NR—C(O)O-substituted heterocyclic wherein R²⁰ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring. Examples of suitable cycloalkyl groups include, for instance, adamantyl, cyclopropyl, cyclobutyl, cyclopentyl, and cyclooctyl. Other examples of cycloalkyl groups include bicycle[2,2,2,]octanyl, norbornyl, and spiro groups such as spiro[4.5]dec-8-yl:

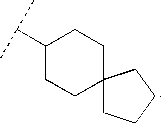

“Cycloalkenyl” refers to non-aromatic cyclic alkyl groups of from 3 to 10 carbon atoms having single or multiple cyclic rings and having at least one >C═C< ring unsaturation and preferably from 1 to 2 sites of >C═C< ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein. “Substituted adamantyl” refers to an adamantyl having from 1 to 5 or preferably 1 to 3 substituents selected from the above list.

“Cycloalkyloxy” refers to —O-cycloalkyl.

“Substituted cycloalkyloxy” refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR²³C(═NR²³)N(R²³)₂ where each R²³ is independently selected from the group consisting of hydrogen, alkyl, substituted alkyl, aryl, substituted aryl, heteroaryl, substituted heteroaryl, heterocyclic, and substituted heterocyclic and two R²³ groups attached to a common guanidino nitrogen atom are optionally joined together with the nitrogen bound thereto to form a heterocyclic or substituted heterocyclic group, provided that at least one R²³ is not hydrogen, and wherein said substituents are as defined herein.

“Halo” or “halogen” refers to fluoro, chloro, bromo and iodo and preferably is fluoro or chloro.

“Haloalkyl” refers to alkyl groups substituted with 1 to 5, 1 to 3, or 1 to 2 halo groups, wherein alkyl and halo are as defined herein.

“Haloalkoxy” refers to alkoxy groups substituted with 1 to 5, 1 to 3, or 1 to 2 halo groups, wherein alkoxy and halo are as defined herein.

“Haloalkylthio” refers to alkylthio groups substituted with 1 to 5, 1 to 3, or 1 to 2 halo groups, wherein alkylthio and halo are as defined herein.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through the non-aromatic ring. In one embodiment, the nitrogen and/or sulfur atom(s) of the heterocyclic group are optionally oxidized to provide for the N-oxide, sulfinyl, or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocyclyl.

“Substituted heterocyclyloxy” refers to the group —O-(substituted heterocyclyl).

“Heterocyclylthio” refers to the group —S-heterocyclyl.

“Substituted heterocyclylthio” refers to the group —S-(substituted heterocyclyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O⁻).

“Spiro ring systems” refers to bicyclic ring systems that have a single ring carbon atom common to both rings.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—. The term “alkylsulfonyl” refers to —SO₂-alkyl. The term “(substituted sulfonyl)amino” refers to —NH(substituted sulfonyl) wherein substituted sulfonyl is as defined herein.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-cycloalkenyl, —OSO₂-substituted cycloalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl. It is also understood that in all substituted groups defined above, polymers arrived at by defining substituents with other substituents (e.g., substituted aryl having a substituted alkyl group as a substituent which is itself substituted with a substituted aryl group, etc.) are not intended to include cases where the maximum number of such substituents exceeds five. That is to say that each of the above definitions is constrained by a limitation that substitutions do not exceed five, for example, substituted aryl groups are limited to -substituted aryl-(substituted alkyl)-(substituted cycloalkyl)-(substituted alkyl)-(substituted alkyl).

Similarly, it is understood that the above definitions are not intended to include impermissible substitution patterns (e.g., methyl substituted with 5 fluoro groups). Such impermissible substitution patterns are well known to the skilled artisan.

“Compound” or “compounds” as used herein is meant to include the racemates, stereoisomers and tautomers of the indicated formulas unless otherwise specified.

“Stereoisomer” or “stereoisomers” include enantiomers and diastereomers and trans- and cis-isomers where applicable. Enantiomers and diastereomers refer to compounds that differ in the chirality at one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Racemates” refers to a mixture of enantiomers.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate. Suitable pharmaceutically acceptable salts also include those listed in Remington's Pharmaceutical Sciences, 17th Edition, pg. 1418 (1985) and P. Heinrich Stahl, Camille G. Wermuth (Eds.), Handbook of Pharmaceutical Salts Properties, Selection, and Use; 2002. Examples of acid addition salts include those formed from acids such as hydroiodic, phosphoric, metaphosphoric, nitric and sulfuric acids, and with organic acids, such as alginic, ascorbic, anthranilic, benzoic, camphorsulfuric, citric, embonic (pamoic), ethanesulfonic, formic, fumaric, furoic, galacturonic, gentisic, gluconic, glucuronic, glutamic, glycolic, isonicotinic, isothionic, lactic, malic, mandelic, methanesulfonic, mucic, pantothenic, phenylacetic, propionic, saccharic, salicylic, stearic, succinic, sulfinilic, trifluoroacetic and arylsulfonic for example benzenesulfonic and p-toluenesulfonic acids. Examples of base addition salts formed with alkali metals and alkaline earth metals and organic bases include chloroprocaine, choline, N,N-dibenzylethylenediamine, diethanolamine, ethylenediamine, lysine, meglumaine (N-methylglucamine), tromethamine, and procaine, as well as internally formed salts.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically-acceptable carrier” encompasses any of the standard pharmaceutical carriers, such as a phosphate-buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

An “excipient” refers to an inert substance added to a pharmaceutical composition to further facilitate administration of the active ingredient.

A “subject,” “individual” or “patient” is used interchangeably herein, and refers to a vertebrate, for example a mammal or preferably a human. Mammals include, but are not limited to, murines, rats, simians, humans, farm animals, sport animals and pets.

An “effective amount” is used synonymously with a “therapeutically effective amount” and intends an amount sufficient to effect beneficial or desired results. An effective amount can be administered in one or more administrations, applications, or dosages. The “effective amount” may vary depending on the compound, the disease and its severity and the age, weight, etc., of the patient to be treated all of which is within the skill of the attending clinician. Generally, therapeutically effective amounts of the compounds may range from approximately 0.05 to 50 mg per kilogram body weight of the recipient per day; commonly about 0.1-25 mg/kg/day, or from about 0.5 to 10 mg/kg/day. Thus, for administration to a 70 kg person, a preferred dosage range would be about 3.5-2000 mg per day.

“Treating” or “treatment” of a disease, disorder or condition will depend on the disease, disorder or condition to be treated and the individual to be treated. In general, treatment intends one or more of (1) inhibiting the progression of the manifested disease, disorder or condition as measured by clinical or sub-clinical parameters (where the term “Inhibiting” or “Inhibition” is intended to be a subset of “Treating” or “treatment”), (2) arresting the development of the disease, disorder or condition as measured by clinical or sub-clinical parameters, (3) ameliorating or causing regression of the disease, disorder or condition as measured by clinical or sub-clinical parameters, or (4) reducing pain or discomfort for the subject as measured by clinical parameters. “Treating” does not include preventing the onset of the disease or condition.

“Preventing” or “prevention” of a disease, disorder or condition means that the onset of the disease or condition in a subject predisposed thereto is prevented such that subject does not manifest the disease, disorder or condition.

### Therapeutic Methods

The present invention in one of its aspect is directed to the use of sEH inhibitors to treat, prevent, or inhibit non-vascular smooth muscle disorders. The present invention is further directed to the surprising and unexpected discovery that use of sEH inhibitors described herein can beneficially enhance smooth muscle function as it relates to the relaxation of smooth muscle. Impairments in non-vascular smooth muscle relaxation are associated with several disorders including, but not limited to, erectile dysfunction, overactive bladder, uterine contractions and irritable bowel syndrome.

“Smooth muscle,” “nonstriated muscle” or “unstriated muscle” is used interchangeably herein, and refers to a tissue that lacks cross striations, which is made up of elongated spindle-shaped cells having a central nucleus and is found in vertebrate hollow organs and structures such as, but not limited to, vascular smooth muscle comprising tunica media layer of arteries and veins and non-vascular smooth muscle including the bladder, uterus, penis, male and female reproductive tracts, gastrointestinal tract, respiratory tract, the ciliary muscle and iris of the eye. This tissue is composed of thin sheets performing functions not subject to direct voluntary control.

“Smooth muscle function” is to maintain dimensions against outside forces. Cells are mechanically coupled to one another such that contraction of one cell invokes some degree of contraction in an adjoining cell. Gap junctions couple adjacent cells chemically and electrically, facilitating the spread of chemicals or action potentials between smooth muscle cells. Smooth muscle may contract spontaneously (via ionic channel dynamic or Cajal pacemaker cells) or be induced by a number of physiochemical agents (e.g., hormones, drugs, neurotransmitters—particularly from the autonomic nervous system), and also mechanical stimulation (such as stretch).

Smooth muscles can be divided into “multi-unit” and “visceral” types or into “phasic” and “tonic” types based on the characteristics of the contractile patterns. Smooth muscles may contract phasically with rapid contraction and relaxation, or tonically with slow and sustained contraction. The reproductive, digestive, respiratory, and urinary tracts, skin, eye, and vasculature all contain this tonic muscle type. By way of example, contractile and relaxation function of vascular smooth muscle is critical to regulating the lumenal diameter of the small arteries-arterioles called resistance vessels. The resistance arteries contribute significantly to setting the level of blood pressure. Smooth muscle contracts slowly and may maintain the contraction for prolonged periods in blood vessels, bronchioles, and some sphincters. By way of another example, in the digestive tract, non-vascular smooth muscle contracts in a rhythmic peristaltic fashion, rhythmically forcing foodstuffs through the digestive tract as the result of phasic contraction.

A “smooth muscle disorder” is characterized by an otherwise healthy smooth muscle which over or under responds to stimuli. Said stimuli are capable of inducing smooth muscle contraction or relaxation as described above. Said stimuli includes, but are not limited to, direct stimulation by the autonomic nervous system, chemical, biological or physical stimulation by neighbouring cells and hormones within the medium that surround the muscle. In some embodiments, the smooth muscle disorder is unrelated to hypertension.

“Erectile dysfunction” (ED) or “male impotence” is characterized by the regular or repeated inability to obtain or maintain an erection. There are several ways that erectile dysfunction is analyzed including, but not limited to:
Obtaining full erections at some times, such as when asleep, when the mind and psychological issues if any are less present, tends to suggest the physical structures are functionally working.
Obtaining erections which are either not rigid or full (lazy erection), or are lost more rapidly than would be expected (often before or during penetration), can be a sign of a failure of the mechanism which keeps blood held in the penis, and may signify an underlying clinical condition.
Other factors leading to erectile dysfunction are diabetes mellitus (causing neuropathy) or hypogonadism (decreased testosterone levels due to disease affecting the testicles or the pituitary gland).

There are many causes of ED and are usually multifactorial in a single subject, including but not limited to, organic, physiologic, endocrine, and psychogenic factors. One of the physiological causes of erectile dysfunction is the inability of the smooth muscle comprising the penis to relax thereby allowing the infiltration of blood into the penis. Disorders which result in the insufficiency or defective relaxation of the smooth muscle can result in ED.

Diseases associated with ED include, but are not limited to; vascular diseases such as atherosclerosis, peripheral vascular disease, myocardial infarction, arterial hypertension, vascular diseases resulting from radiation therapy or prostate cancer treatment, blood vessel and nerve trauma; systemic diseases such as diabetes mellitus, scleroderma, renal failure, liver cirrhosis, idiopathic hemochromatosis, cancer treatment, dyslipidemia and hypertension; neurogenic diseases such as, epilepsy, stroke, multiple sclerosis, Guillain-Barré syndrome, Alzheimers disease and trauma; respiratory diseases such as, chronic obstructive pulmonary disease and sleep apnea; hematologic diseases such as sickle cell anemia and leukemias; endocrine conditions such as, hyperthyroidism, hypothyroidism, hypogonadism and diabetes; penile conditions such as, peyronie disease, epispadias and priapism; and psychiatric conditions such as depression, widower syndrome, performance anxiety and posttraumatic stress disorder. Additional states which are associated with ED include nutritional states such as, malnutrition and zinc deficiency; surgical procedures such as, procedures on the brain and spinal cord, retroperitoneal or pelvic lymph node dissection, aortioliac or aortofemoral bypass, abdominal perineal resection, surgical removal of the prostate, proctocolectomy, transurethral resection of the prostate, and cryosurgery of the prostate; and treat with medication such as, antidepressants, antipsychotics, antihypertensives, antiulcer agents, 5-alpha reductase inhibitors and cholesterol-lowering agents.

“Overactive bladder” (OAB) is defined by the International Continence Society as a urological condition defined by a set of symptoms: urgency, with and without urge incontinence, usually with frequency and nocturia. The etiology of OAB is still unclear, however it is often associated with detrusuor overactivity, a pattern of bladder muscle contraction observed during urodynamic.

“Irritable bowel syndrome” (IBS) also known as “spastic colon” is a functional bowel disorder characterized by abdominal pair and altered bowel habits in the absence of specific and unique organic pathology. IBS is a clinically defined disease, wherein one set of criteria is that the subject must have recurrent abdominal pain or discomfort at least 3 days per month during the previous 3 months that is associated with 2 or more of the following: relieved by defecation, onset associated with a change in stool frequency and onset associated with a change in stool form or appearance. Additional symptoms included altered stool frequency, altered stool form, altered stool passage (straining and/or urgency), mucorrhea and abdominal bloating or subjective distention.

“Non-inflammatory irritable bowel syndrome” refers to IBS with no signs or symptoms of inflammation of the colon.

“Uterine Contraction” is the tightening and shortening of the smooth muscles comprising the uterus. Irregular contractions, increased frequency or increased contraction strength of the uterus can be associated with the pre-menstrual syndrome (PMS) or during premature or normal labor delivery of a fetus.

Accordingly, in one aspect the invention provides a method for enhancing smooth muscle function by administering to the subject predisposed to or having a disorder, disease or condition associated therewith an effective amount of a sEH inhibitor. The method is unrelated to hypertension. In a further aspect, the method enhances the smooth muscle relaxation of non-vascular smooth muscle. This non-vascular smooth muscle in some aspects comprises the male or female reproductive tract, bladder or gastrointestinal tract of said subject.

In another aspect, the invention provides a method for treating a non-vascular smooth muscle disorder in a subject, wherein the smooth muscle disorder is characterized by an otherwise healthy smooth muscle which over or under responds to stimuli by administering to the subject an effective amount of a sEH inhibitor. In one embodiment, the smooth muscle disorder is not hypertension. In yet a further aspect the subject is suffering from a smooth muscle disorder selected from, but not limited to, erectile dysfunction, overactive bladder, uterine contractions, irritable bowel syndrome, non-inflammatory irritable bowel syndrome, general gastrointestinal tract motility.

In a further aspect of the above embodiments, a subject is unable to be treated with an effective amount of a phosphodiesterase type 5 inhibitor. Examples of phosphodiesterase type 5 inhibitors include, but are not limited to, sildenafil, tadalafil, vardenafil, udenafil and avanafil. In a further aspect, the subject of the above embodiments are unable to be treated with a phosphodiesterase type 5 inhibitor due to a preexisting disease, disorder or condition including, but not limited to, congestive heart failure, heart disease, stroke, hypotension, diabetes or any combination thereof.

In a further aspect of the above embodiments, a subject is unable to be treated with an effective amount of an anticholinergic. Examples of anticholinergics include, but are not limited to, dicycloverine, tolterodine, oxybutynin, trospium and solifenacin.

In another aspect of this invention, provided is a method of treating a vascular smooth muscle disorder in a patient in need thereof, comprising administering to said patient a therapeutically effective amount of a compound, which includes:
1-adamantyl-3-(1-(methyl sulfonyl)piperidin-4-yl)urea;
1-(1-nicotinoylpiperidin-4-yl)-3-(4-(trifluoromethoxy)phenyl)urea; or
1-adamantyl-3-(1-acetylpiperidin-4-yl)urea.

These compounds are sEH inhibitors.

### sEH Inhibitors

In each of the above embodiments, the activity of soluble epoxide hydrolase is inhibited. Inhibition of can be accomplished by any of the methods available to and known by those of skill in the art. In some embodiments, an effective amount of a sEH inhibitor, or composition comprising a sEH inhibitor, is administered to a subject in need thereof. Preferably, the sEH inhibitor is biocompatible and pharmaceutical compounds.

sEH inhibitors are well known in the art and include but are not limited to those disclosed in McElroy et al, J. Med. Chem., 46:1066-1080 (2003); U.S. Pat. Nos. 6,831,082, and 6,693,130, US Patent Application Publications 2007/0225283, 2006/0270609, 2008/0076770, 2008/0032978, 2008/153889, 2008/0207621, 2008/0207622, 2008/0200444, 2008/0200467, 2008/0227780, 2009/0023731, 2009/0082395, 2009/0082350, 2009/0082456, and 2009/0082423, U.S. Provisional Patent Application 61/046,316, filed on Apr. 18, 2008, and International patent applications WO2008/105968, WO2007/043652, WO2007/043653, WO2007/106705, WO2007/067836, WO2007/098352, WO2008/022171, WO2006/121719, WO2007/044491, WO2006/121684, WO2009/011872, WO2009/020960, and PCT/US2008/088244. All of the above listed publications, patents, patent applications are incorporated by reference in their entirety.

In some embodiments, the sEH inhibitors are described by at least one of the following general or specific formulas shown in Formula (A), (I), to Formula (V) or in Tables 1 and 2.

In some embodiments, the compound is of Formula (A), or a stereoisomer, tautomer, or pharmaceutically acceptable salt thereof:

R¹-L-C(=Q)NR²R^{2a} (A)

wherein:
L is selected from the group consisting of —NH—, —CR^{a}R^{b}—, a covalent bond, and —CR^{a}R^{b}NH—, where R^{a} and R^{b} are independently hydrogen or alkyl, or R^{a} and R^{b} together with the carbon bound thereto form a C₃-C₆ cycloalkyl;
Q is selected from the group consisting of O and S; and
R¹ and R² are independently selected from the group consisting of substituted alkyl, aryl, substituted aryl, heteroaryl, substituted heteroaryl, cycloalkyl, substituted cycloalkyl, heterocycloalkyl, and substituted heterocycloalkyl;
R^{2a} is selected from the group consisting of hydrogen, and alkyl;
or R² and R^{2a} together with the nitrogen bound thereto form an optionally substituted heterocycloalkyl;
provided that when L is not —NH—, R^{2a} is hydrogen.

In some embodiments, L is —NH—. In some embodiments, L is —CR^{a}R^{b}. In some embodiments, L is —CR^{a}R^{b}NH—. In some embodiments, L is a covalent bond.

In some embodiments, R² and R^{2a} together with the nitrogen bound thereto form an optionally substituted heterocycloalkyl.

In one aspect, the compound is a member of the group of Formula (I):

R¹NHC(=Q)NHR² (I)

wherein:
Q is selected from the group consisting of O and S; and
R¹ and R² are independently selected from the group consisting of substituted alkyl, aryl, substituted aryl, heteroaryl, substituted heteroaryl, cycloalkyl, substituted cycloalkyl, heterocycloalkyl, and substituted heterocycloalkyl; or
a stereoisomer, tautomer, or pharmaceutically acceptable salt thereof.

In a further aspect, the R¹ is cycloalkyl, substituted cycloalkyl, phenyl or substituted phenyl. In a further aspect, the R¹ is cycloalkyl or cycloalkyl substituted with 1 to 4 alkyl groups. In one aspect, the cycloalkyl is selected from the group consisting of cyclohexyl, cycloheptyl, cyclooctyl, and adamantyl. In another aspect, R¹ is substituted phenyl, for example phenyl substituted at least one substituent selected from the group consisting of halo, haloalkyl and haloalkoxy.

In a further aspect, the R² is substituted alkyl or substituted heterocycloalkyl. In a further aspect, the R² is substituted phenyl.

In one embodiment, R² is substituted heterocycloalkyl. In one embodiment, heterocycloalkyl is containing one or more nitrogen as a hetero atom. In another embodiment, R² is

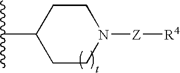

wherein
Z is CO, SO, SO₂, or a covalent bond;
R⁴ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl; and
t is an integer equal to 0, 1, or 2.

In one aspect, the compound is a member of the group of Formula (II) or (III):

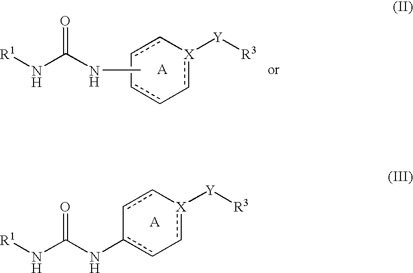

wherein:
R¹ is selected from the group consisting of aryl, substituted aryl, heteroaryl, substituted heteroaryl, cycloalkyl, substituted cycloalkyl, heterocycloalkyl, and substituted heterocycloalkyl;
X is CH, C or N; provided that when X is CH then ring A is cyclohexyl, when X is C then ring A is phenyl, and when X is N then ring A is piperidinyl;
Y is selected from the group consisting of CO, a covalent bond, O, and SO₂; and
R³ is selected from the group consisting of alkyl, substituted alkyl, heteroaryl, substituted heteroaryl, heterocycloalkyl and substituted heterocycloalkyl; or
a stereoisomer, tautomer, or pharmaceutically acceptable salt thereof.

In one aspect, the compound is a member of the group of Formula (IV):

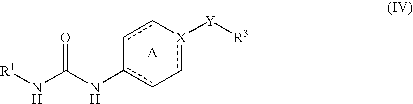

wherein:
R¹ is selected from the group consisting of aryl, substituted aryl, heteroaryl, substituted heteroaryl, cycloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂; and
R³ is selected from the group consisting of alkyl, substituted alkyl heteroaryl, substituted heteroaryl, heterocycloalkyl and substituted heterocycloalkyl; or
a stereoisomer, tautomer, or pharmaceutically acceptable salt thereof.

In one aspect, R¹ is cyclohexyl or substituted cyclohexyl. In a further aspect, R¹ is adamantyl or substituted adamantyl.

In one aspect, R¹ is phenyl. In another aspect, R¹ is substituted phenyl.

In one aspect, the compound is a member of the group of Formula (V):

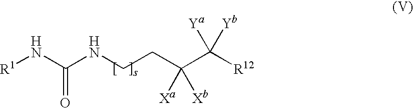

wherein
R¹ is selected from the group consisting of aryl, substituted aryl, heteroaryl, substituted heteroaryl, cycloalkyl, substituted cycloalkyl, heterocycloalkyl, and substituted heterocycloalkyl;
s is 0-10;
R¹² is selected from the group consisting of —OR¹³, —CH₂OR¹³, —COR¹³, —COOR¹³, —CONR¹³R¹⁴, and carboxylic acid isostere; and
R¹³ and R¹⁴ are independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; or R¹³ and R¹⁴ together with the nitrogen atom bound thereto form a heterocycloalkyl ring having 3 to 9 ring atoms, and wherein said ring is optionally substituted with alkyl, substituted alkyl, heterocycloalkyl, oxo or carboxy; and
each of X^{a}, X^{b}, Y^{a}, and Y^{b} is independently selected from the group consisting of hydrogen, C₁-C₄ alkyl, substituted C₁-C₄ alkyl, and halo, provided that at least one of Y^{a} and Y^{b} is halo or C₁-C₄ alkyl;
or a stereoisomer, tautomer, or pharmaceutically acceptable salt thereof.

In one aspect, R¹ is cyclohexyl or substituted cyclohexyl. In a further aspect, R¹ is adamantyl or substituted adamantyl.

In yet one aspect, R¹ is phenyl. In another aspect, R¹ is substituted phenyl.

In yet one aspect, R¹² is selected from the group consisting of —CH₂OR¹³, —COR¹³, —COOR¹³, —CONR¹³R¹⁴, and carboxylic acid isostere. In yet one aspect, R¹² is selected from the group consisting of —CH₂OR¹³, —COR¹³, —COOR¹³, and —CONR¹³R¹⁴.

In still yet a further aspect, at least one of Y^{a} and Y^{b} is halo.

In another embodiment, the compound to be administered is a compound, a stereoisomer, tautomer, or pharmaceutically acceptable salt thereof to a compound selected from Tables 1 and 2.

TABLE 1

| Compound | | |
|---|---|---|
| No. | Structure | Name |
| 1 | 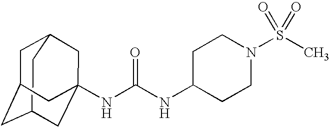 | 1-adamantyl-3-(1- (methylsulfonyl)piperidin-4-yl)urea |
| 2 | 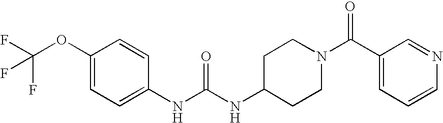 | 1-(1-nicotinoylpiperidin-4-yl)-3-(4- (trifluoromethoxy)phenyl)urea |
| 3 | 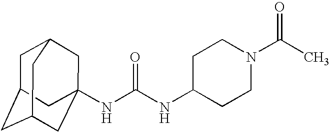 | 1-adamantyl-3-(1-acetylpiperidin-4- yl)urea |
| 4 | 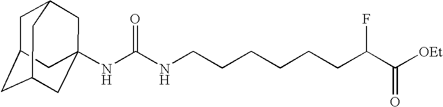 | ethyl 2-fluoro-8-(3- adamantylureido)octanoate |
| 5 | 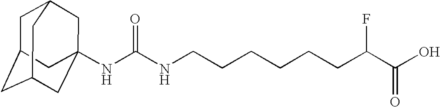 | 2-fluoro-8-(3-adamantylureido)octanoic acid |

TABLE 2

| | |
|---|---|
| 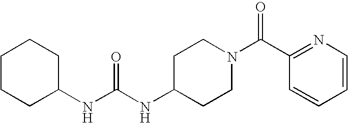 | 1-cyclohexyl-3-(1-picolinoylpiperidin-4- yl)urea |
| 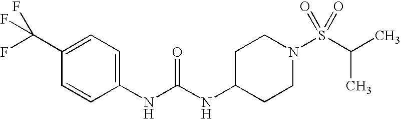 | 1-(1-(isopropylsulfonyl)piperidin-4-yl)-3- (4-(trifluoromethyl)phenyl)urea |
| 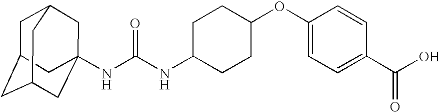 | 4-(4-3-admantan-1-yl- ureido)cyclohexyloxy)benzoic acid |
| 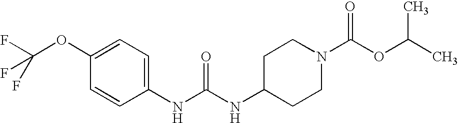 | isopropyl 4-(3-(4- (trifluoromethoxy)phenyl)ureido)piperidine- 1-carboxylate |
| 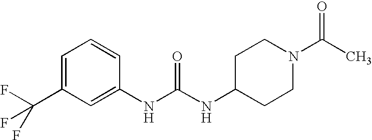 | 1-(1-acetyl-piperidin-4-yl)-3-(3- trifluoromethyl-phenyl)-urea |
| 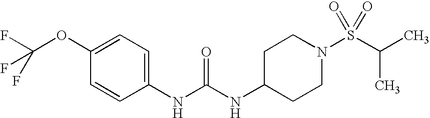 | 1-(1-(isopropylsulfonyl)piperidin-4-yl)-3- (4-(trifluoromethoxy)phenyl)urea |
| 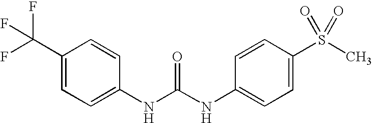 | 1-(4-methanesulfonyl-phenyl)-3-(4- trifluoromethyl-phenyl)-urea |
| 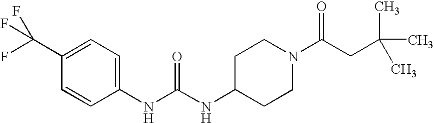 | 1-(1-(3,3-dimethylbutanoyl)piperidin-4-yl)- 3-(4-(trifluoromethyl)phenyl)urea |
| 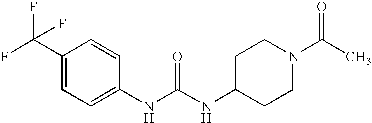 | 1-(1-acetyl-piperidin-4-yl)-3-(4- trifluoromethyl-phenyl)-urea |
| 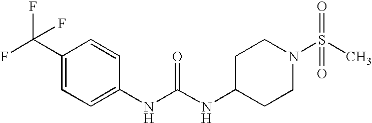 | 1-(1-methanesulfonyl-piperidin-4-yl)-3-(4- trifluoromethyl-phenyl)-urea |

The compounds listed above can be referred to by their compound number or an alternative name. For example, 1-adamantyl-3-(1-(methylsulfonyl)piperidin-4-yl)urea can be referred to as Compound 1 or, alternatively, 1-[1-(methylsulfonyl)piperidin-4-yl]-N′-(adamant-1-yl)urea. Likewise, 1-adamantyl-3-(1-acetylpiperidin-4-yl)urea can be referred to as Compound 3 or, alternatively, N-(1-acetylpiperidin-4-yl)-N′-(adamant-1-yl)urea.

Other means of inhibiting sEH activity or expression can also be used. Non-limiting examples include siRNA, dsRNA, miRNA, antisense polynucleotide, ribozymes, triplex polynucleotide, antibody and other inhibitory polypeptides. General means of preparing siRNA, dsRNA, miRNA, antisense polynucleotide, ribozymes, triplex polynucleotide, antibody and other polypeptides having sEH inhibitory activity are known the art. U.S. Provisional Patent Application No. 61/151,493 is incorporated herein by reference in its entirety.

In another aspect of the invention, one or more of sEH inhibitors or pharmaceutically acceptable salts thereof, may be used in the preparation of a medicament for the treatment of a smooth muscle disorder or a smooth muscle disorder selected from one or more of the following: erectile dysfunction, overactive bladder, uterine contractions or irritable bowel syndrome.

### Compositions and Formulations

The compositions are comprised of in general, a sEH inhibitor in combination with at least one pharmaceutically acceptable carrier or excipient. Acceptable carriers are known in the art and described supra. Acceptable excipients are non-toxic, aid administration, and do not adversely affect the therapeutic benefit of the compound. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols.

The sEH inhibitors can be administered in any suitable formulation such as a tablet, pill, capsule, semisolid, gel, transdermal patch or solution, powders, sustained release formulation, solution, suspension, elixir or aerosol. The most suitable formulation will be determined by the disease or disorder to be treated and the individual to be treated.

Compressed gases may be used to disperse a sEH inhibitor of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The following are representative pharmaceutical formulations containing a sEH inhibitor of the present invention.

### Tablet Formulation

The following ingredients are mixed intimately and pressed into single scored tablets.

| Ingredient | Quantity per tablet, mg |
|---|---|
| sEH inhibitor | 400 |
| Cornstarch | 50 |
| Croscarmellose sodium | 25 |
| Lactose | 120 |
| Magnesium stearate | 5 |

### Capsule Formulation

The following ingredients are mixed intimately and loaded into a hard-shell gelatin capsule.

| Ingredient | Quantity per capsule, mg |
|---|---|
| sEH inhibitor | 200 |
| Lactose, spray-dried | 148 |
| Magnesium stearate | 2 |

### Injectable Formulation

The following ingredients are mixed to form an injectable formulation.

| Ingredient | Quantity per injection, mg |
|---|---|
| sEH inhibitor | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Suspension Formulation

The following ingredients are mixed to form a suspension for oral administration (q.s.=sufficient amount).

| Ingredient | Amount | |
|---|---|---|
| sEH inhibitor | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.0 | g |
| Sorbitol (70% solution) | 13.0 | g |
| Veegum K (Vanderbilt Co) | 1.0 | g |
| Flavoring | 0.035 | mL |
| colorings | 0.5 | mg |
| distilled water | q.s. to 100 | mL |

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Quantity per suppository, mg |
|---|---|
| sEH inhibitor | 500 mg |
| Witepsol ® H-15 | balance |

Also provided is a medicament comprising a compound or composition as described herein for use in treating a disease or disorder as described above, which can be identified by noting any one or more clinical or sub-clinical parameters.

### Combination Therapy

For more generalized therapeutic purposes, combination therapy is often desirable. Combination therapy includes administration of a single pharmaceutical dosage formulation which contains a sEH inhibitor and one or more additional active agents, or therapies such as heat, light and such, as well as administration of the sEH inhibitor and each active agent in its own separate pharmaceutical dosage formulation. For example, a compound of this invention and one or more of other agents including, but not limited to, COX2 inhibitors, PDE5 inhibitors angiotensin concerting enzyme inhibitors, angiotensin II receptor blockers could be administered to the human subject together in a single oral dosage composition such as a tablet or capsule or each agent can be administered in separate oral dosage formulations. Other useful agents in the treatment of the smooth muscle disorders include anticholinergics. Combination therapy is understood to include all these regimens.

### Dosing and Administration

The present invention provides therapeutic methods generally involving administering to a subject in need thereof an effective amount of sEH inhibitors described herein. The dose, frequency, and timing of such administering will depend in large part on the selected therapeutic agent, the nature of the condition to be treated, the condition of the subject, including age, weight and presence of other conditions or disorders, the formulation of the therapeutic agent and the discretion of the attending physician. The sEH inhibitors and compositions described herein and the pharmaceutically acceptable salts thereof are administered via oral, parenteral, subcutaneous, intramuscular, intravenous or topical routes. Generally, it is contemplated that the sEH inhibitors are to be administered in dosages ranging from about 0.10 milligrams (mg) up to about 1000 mg per day, although variations will necessarily occur, depending, as noted above, on the target tissue, the subject, and the route of administration. In preferred embodiments, the sEH inhibitors are administered orally once or twice a day.

The sEH inhibitors may be administered in a range of from approximately 0.05 to 50 mg per kilogram body weight of the recipient per day; often about 0.1 to 25 mg/kg/day, more often from about 0.5 to 10 mg/kg/day. An sEH inhibitor may be administered to a human subject in a range between about 0.10 mg and 2000 mg per day, or in some embodiments, the compounds are administered in a range between about 1 mg and 800 mg per day, or between about 2 mg and 600 mg per day, or between about 5 mg and 500 mg per day, or between about 10 mg and 200 mg per day; or in some embodiments, the compounds are administered in a range between about 50 mg and 100 mg per day.

The following examples are provided to illustrate certain aspects of the present invention and to aid those of skill in the art in practicing the invention. These examples are in no way to be considered to limit the scope of the invention.

### Synthetic Chemistry

The sEH inhibitors of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the sEH inhibitors of this invention may contain one or more chiral centers. Accordingly, if desired, such inhibitors can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Preferably, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds of the invention may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

Scheme 1 below illustrates a general synthetic method for the preparation of the compounds of Formula (I).

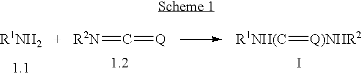

R¹NH₂+R²N═C=Q→R¹NH(C=Q)NHR² 1.1 1.2 I Scheme 1

A synthesis of the compounds of the invention is shown in Scheme 1, where Q, R¹, and R² are as previously defined. Specifically, amine 1.1 reacts with the appropriate isocyanate or thioisocyanate 1.2 to form the corresponding urea or thiourea of Formula (I). Typically, the formation of the urea is conducted using a polar solvent such as DMF (dimethylformamide) at 0 to 10° C. Isocyanate or thioisocyanate 1.2 can be either known compounds or can be prepared from known compounds by conventional synthetic procedures. Suitable isocyanates include by way of example only, adamantyl isocyanate, cyclohexyl isocyanate, phenyl isocyanate, trifluoromethylphenyl isocyanate, chlorophenyl isocyanate, fluorophenyl isocyanate, trifluoromethoxyphenyl isocyanate and the like.

Scheme 2 illustrates the methods of Scheme 1 as they relate to the preparation of piperidinyl compounds of Formula (II).

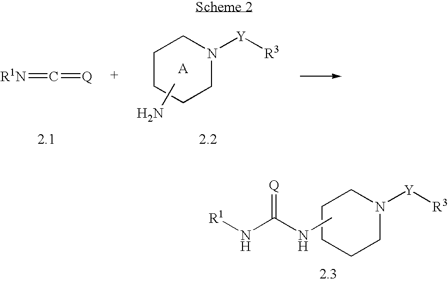

Scheme 2 can also be employed for the synthesis of compounds of Formula (II) where, for illustrative purposes, ring A is a piperidinyl ring and Q, Y, R¹, R³, and m are previously defined. Reaction of 2.1 with amine 2.2 forms the corresponding urea or thiourea of 2.3.

In Scheme 2, the N—(YR³) substituted piperidinyl amine can be prepared as shown in Scheme 3 below:

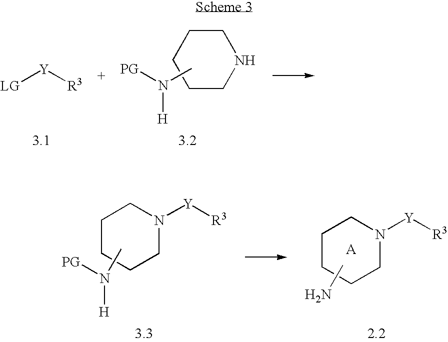

Where Y and R³ are as defined above and LG is a leaving group such as a halo group, a tosyl group, a mesyl group, and the like and PG is a conventional amino protecting group such as a tert-butoxycarbonyl (Boc) group. Reaction of 3.1 with protected aminopiperidine 3.2 forms the functionalized amine 3.3. Removal of the protecting group gives 2.2. Both of these reactions are conventional and well within the skill of the art.

The following schemes illustrate preferred methods of preparing certain compounds of Formula (I) and/or (II). Specifically, in Scheme 4, a 4-amidopiperidine group is employed for illustrative purposes only and this scheme illustrates the synthesis of N-(1-acylpiperidin-4-yl)-N′-(adamant-1-yl)urea compounds:

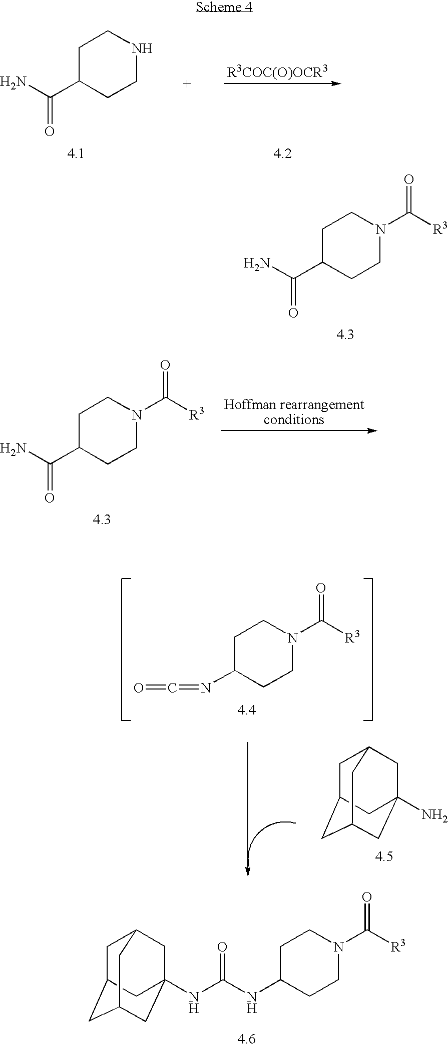

where R³ is defined herein.

In Scheme 4, the amino group of compound 4.1 is acylated using conventional conditions. Specifically, a stoichiometric equivalent or slight excess of a carboxylic acid anhydride 4.2 (which is used only for illustrative purposes) is reacted with compound 4.1 in the presence of a suitable inert diluent such as tetrahydrofuran, chloroform, methylene chloride and the like. When an acid chloride is employed in place of the acid anhydride, the reaction is typically conducted in the presence of an excess of a suitable base to scavenge the acid generated during the reaction. Suitable bases are well known in the art and include, by way of example only, triethylamine, diisopropylethylamine, pyridine, and the like.

The reaction is typically conducted at a temperature of from about 0 to about 40° C. for a period of time sufficient to effect substantial completion of the reaction which typically occurs within about 1 to about 24 hours. Upon reaction completion, the acylpiperidylamide, compound 4.3, can be isolated by conventional conditions such as precipitation, evaporation, chromatography, crystallization, and the like or, alternatively, used in the next step without isolation and/or purification. In certain cases, compound 4.3 precipitates from the reaction.

Compound 4.3 is then subjected to Hoffman rearrangement conditions to form isocyanate compound 4.4 under conventional conditions. In certain cases, Hoffman rearrangement conditions comprise reacting with an oxidative agent preferably selected from (diacetoxyiodo)benzene, base/bromine, base/chlorine, base/hypobromide, or base/hypochloride. Specifically, approximately stoichiometric equivalents of the N-acyl-4-amidopiperidine, compound 4.3, and, e.g., (diacetoxyiodo)benzene are combined in the presence of a suitable inert diluent such as acetonitrile, chloroform, and the like. The reaction is typically conducted at a temperature of from about 40° C., to about 100° C., and preferably at a temperature of from about 70° C., to about 85° C., for a period of time sufficient to effect substantial completion of the reaction which typically occurs within about 0.1 to about 12 hours. Upon reaction completion, the intermediate isocyanate, compound 4.4, can be isolated by conventional conditions such as precipitation, evaporation, chromatography, crystallization, and the like.

Alternatively and preferably, this reaction is conducted in the presence of adamantyl amine, compound 4.5, such that upon formation of the isocyanate, compound 4.4, the isocyanate functionality of this compound can react in situ with the amino functionality of compound 4.5 to provide for compound 4.6. In this embodiment, the calculated amount of the intermediate isocyanate is preferably employed in excess relative to the adamantyl amine and typically in an amount of from about 1.1 to about 1.2 equivalents based on the number of equivalents of adamantyl amine employed. The reaction conditions are the same as set forth above and the resulting product can be isolated by conventional conditions such as precipitation, evaporation, chromatography, crystallization, and the like.

Compound 4.4 is a stable intermediate. In certain cases, compound 4.4 is formed substantially free from impurities. Hence, Scheme 4 can be run as telescoping reaction processes.

Scheme 5 below illustrates an alternative synthesis of a urea compound where again a 4-amidopiperidine is employed for illustrative purposes:

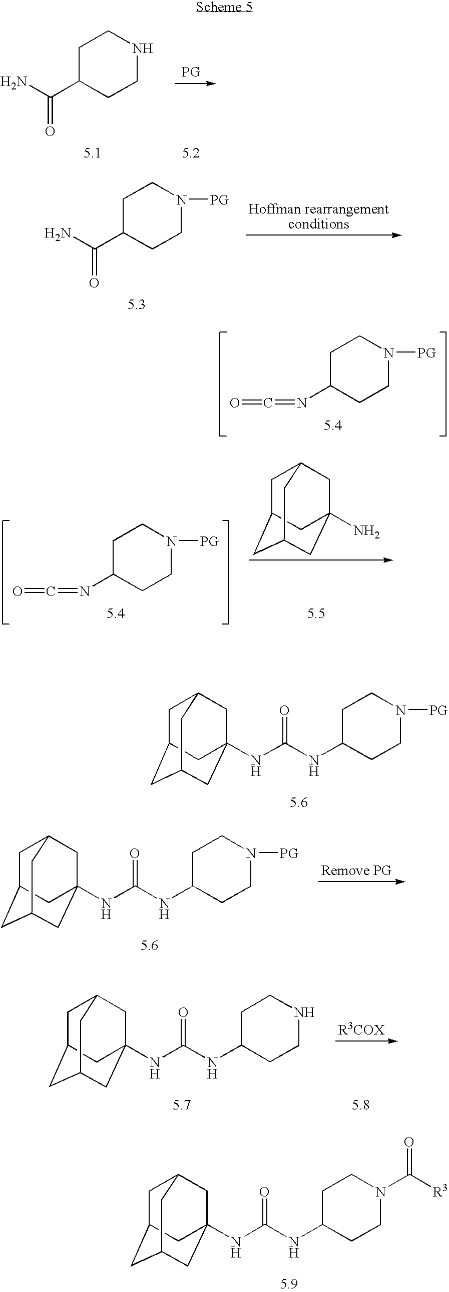

where R³ and PG are as defined herein and X is selected from the group consisting of OH, halo and —OC(O)R³.

Specifically, in Scheme 5, coupling of the adamantyl urea to the piperidinyl ring occurs prior to acylation of the piperidinyl nitrogen atom. In Scheme 5, the amine functionality of compound 5.1 is protected using a conventional amino protecting group (PG) which is well known in the art. In certain cases, the amino protecting group is a benzyl protecting group which can be derived from benzyl chloride and benzyl bromide. Compound 5.3 is subjected to Hoffman rearrangement conditions to form isocyanate compound 5.4 in the manner described in detail above. Compound 5.4 is a stable intermediate. The reaction of compound 5.4 with adamantyl amine is conducted as per Scheme 4 and is preferably conducted in a single reaction step wherein intermediate compound 5.4 is reacted in situ with adamantyl amine, compound 5.5, to form compound 5.6. Compound 5.6 is subjected to conditions to remove the protecting group to yield compound 5.7. In certain cases, the protecting group is benzyl and the removal conditions are palladium-carbon with methanol and formic acid. Compound 5.7 is acylated with compound 5.8 to form compound 5.9 as per Scheme 4 above.

Scheme 6 below illustrates the synthesis of N-(1-alkylsulfonylpiperidin-4-yl)-N′-(adamant-1-yl)ureas:

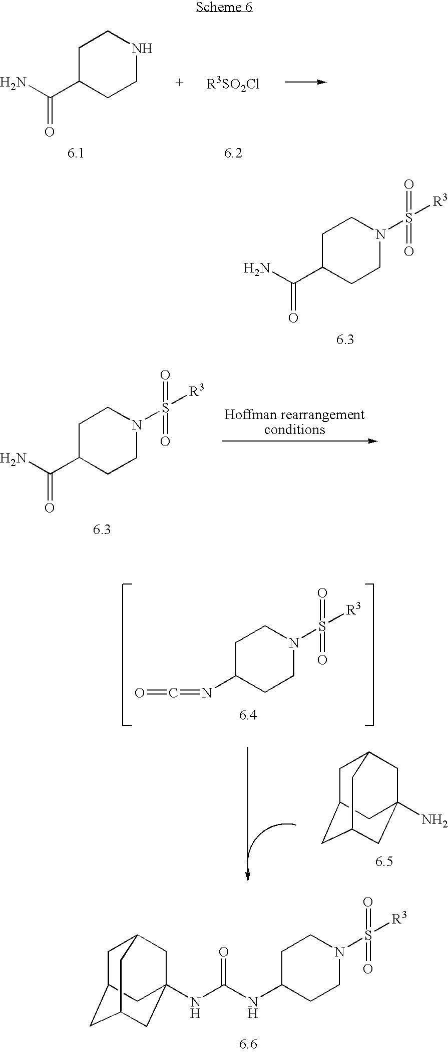

wherein R³ is defined herein.

Specifically, in Scheme 6, amino compound 6.1 is reacted with a sulfonyl halide, compound 6.2 (used for illustrative purposes only), to provide for sulfonamide compound 6.3. This reaction is typically conducted by reacting the compound 6.1 with at least one equivalent, preferably about 1.1 to about 2 equivalents, of the sulfonyl halide (for illustrative purposes depicted as the sulfonyl chloride) in an inert diluent such as dichloromethane, chloroform and the like. Generally, the reaction is preferably conducted at a temperature ranging from about −10° C. to about 20° C. for about 1 to about 24 hours. Preferably, this reaction is conducted in the presence of a suitable base to scavenge the acid generated during the reaction. Suitable bases include, by way of example, tertiary amines, such as triethylamine, diisopropylethylamine, N-methylmorpholine and the like. Alternatively, the reaction can be conducted under Schotten-Baumann-type conditions using aqueous alkali, such as sodium hydroxide and the like, as the base. Upon completion of the reaction, the resulting sulfonamide, compound 6.3, is recovered by conventional methods including neutralization, extraction, precipitation, chromatography, filtration, and the like or, alternatively, used in the next step without purification and/or isolation.

Compound 6.3 is subjected to Hoffman rearrangement conditions as described above to form isocyanate compound 6.4. The reaction of compound 6.4 with adamantyl amine, compound 6.5, is conducted as per Scheme 4 and is preferably conducted in a single reaction step wherein the isocyanate, compound 6.4, is reacted in situ with adamantyl amine, compound 6.5, to form compound 6.6.

The sulfonyl chlorides employed in the above reaction are also either known compounds or compounds that can be prepared from known compounds by conventional synthetic procedures. Such compounds are typically prepared from the corresponding sulfonic acid, using phosphorous trichloride and phosphorous pentachloride. This reaction is generally conducted by contacting the sulfonic acid with about 2 to 5 molar equivalents of phosphorous trichloride and phosphorous pentachloride, either neat or in an inert solvent, such as dichloromethane, at temperature in the range of about 0° C. to about 80° C. for about 1 to about 48 hours to afford the sulfonyl chloride. Alternatively, the sulfonyl chloride can be prepared from the corresponding thiol compound, i.e., from compounds of the formula R³—SH where R³ is as defined herein, by treating the thiol with chlorine (Cl₂) and water under conventional reaction conditions.

Compound 6.4 is a stable intermediate. In certain cases, compound 6.3 is formed substantially free from impurities. Hence Scheme 6 can be run as a telescoping reaction processes.

Scheme 7 below illustrates an alternative synthesis of a urea compound.

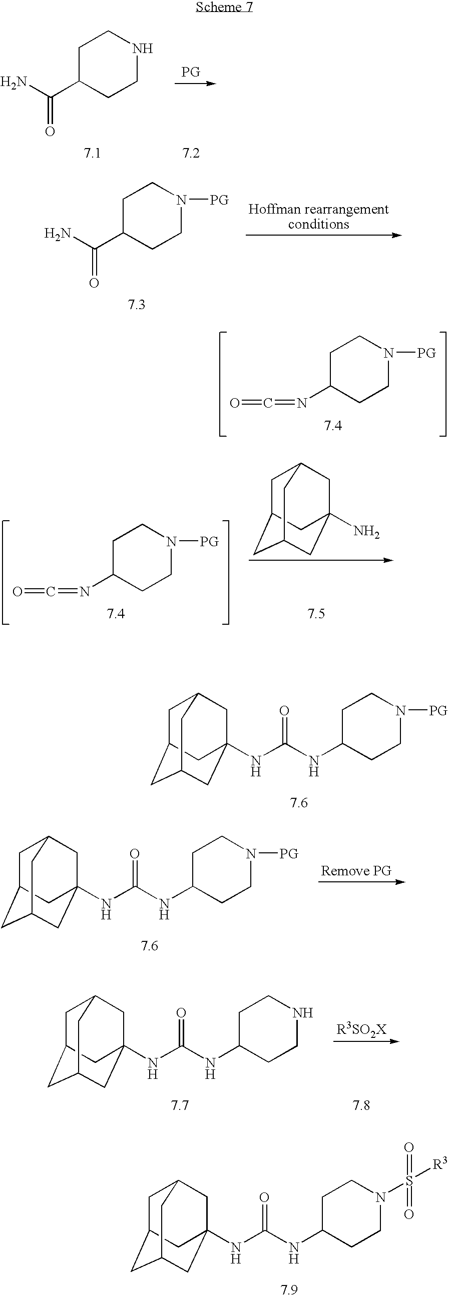

wherein R³, X and PG are defined herein.

Specifically, in Scheme 7, coupling of the adamantyl urea, compound 7.5, to the piperidinyl ring occurs prior to sulfonylation of the piperidinyl nitrogen atom. In Scheme 7, the amine functionality of compound 7.1 is protected using a conventional amino protecting group (PG) which is well known in the art. In certain cases, the amino protecting group is a benzyl protecting group which can be derived from benzyl chloride or benzyl bromide. Compound 7.3 is subjected to Hoffman rearrangement conditions to form isocyanate compound 7.4 in the manner described in detail above. Compound 7.4 is a stable intermediate. The reaction of compound 7.4 with adamantyl amine, compound 7.5, is conducted as per Scheme 4 and is preferably conducted in a single reaction step wherein intermediate compound 7.4 is reacted in situ with adamantyl amine, compound 7.5, to form compound 7.6. Compound 7.6 is subjected to conditions to remove the protecting group to yield compound 7.7. In certain cases, the protecting group is benzyl and the removal conditions are palladium-carbon with methanol and formic acid. Compound 7.7 is then sulfonylated with compound 7.8 to form compound 7.9 as per Scheme 6 above.

The following schemes illustrate preferred methods of preparing compounds of Formula (I) and/or (V) represented by compound 8.2 (Scheme 8).

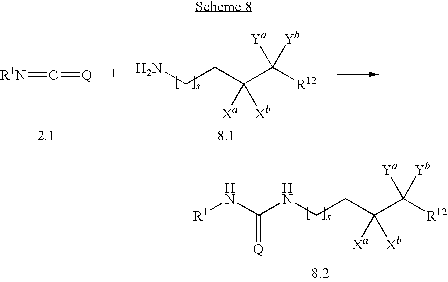

Specifically, as depicted in Scheme 9, an ethyl amino-2-fluoroalk-2-enoate 9.1 is employed for illustrative purposes only:

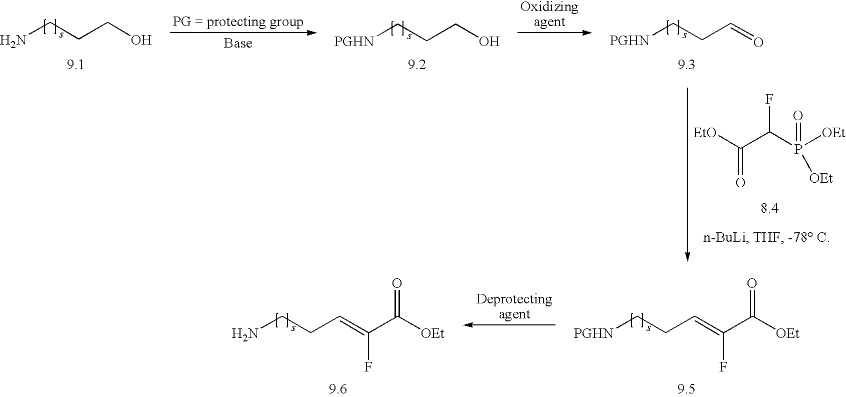

In Scheme 9, s is as defined herein. The synthesis of the compounds of the invention can be exemplified by, but is not limited to, the preparation of the intermediate 9.6, as shown in Scheme 9. Amine 9.1 can be protected with any amine protecting group known in the art (for example, 2,4-dimethoxy-benzyl (DMB), tert-butoxycarbonyl (Boc) etc.) to give compounds 9.2. For example, amine 9.1 can be treated with t-Boc anhydride in the presence of a base, such as sodium carbonate, and a suitable solvent such as, THF to give compounds 9.2. Upon reaction completion, 9.2 can be recovered by conventional techniques such as neutralization, extraction, precipitation, chromatography, filtration and the like; or, alternatively, used in the next step without purification and/or isolation.

Compounds 9.2 are then treated with any suitable oxidizing agent known in the art, to give aldehydes 9.3. For example, 9.2 can be treated with pyridinium chlorochromate (PCC) and neutral alumina (Al₂O₃) in the presence of a suitable solvent, such as, dichloromethane (DCM) to give 9.3. Upon reaction completion, 9.3 can be recovered by conventional techniques such as neutralization, extraction, precipitation, chromatography, filtration and the like; or, alternatively, used in the next step without purification and/or isolation.

Compounds 9.3 are then treated with triethyl-2-fluoro-2-phosphonoacetate 9.4 to give compounds 9.5. This is typically performed in dry tetrahydrofuran (THF) or another suitable solvent known to one skilled in the art, typically at, but not limited to, room temperature in the presence of n-butyllithium (n-BuLi), or another suitable base known to one skilled in the art. Upon reaction completion, 9.5 can be recovered by conventional techniques such as neutralization, extraction, precipitation, chromatography, filtration and the like; or, alternatively, used in the next step without purification and/or isolation.

Compounds 9.5 are then deprotected using a suitable deprotecting agent known in the art to give the intermediate 9.6. For example, deprotection can be achieved, in addition to other methods known to one skilled in the art, by treatment of 9.5 with SOCl₂ in a suitable solvent such as dichloromethane (DCM) (preferred method for PG=2,4-dimethoxy-benzyl (DMB)). Alternatively, 9.5 can be deprotected with TFA neat or in a suitable solvent known to one skilled in the art such as, DCM to give the compounds 9.6 (preferred method for PG=tert-butoxycarbonyl (Boc)). Upon reaction completion, 9.6 can be recovered by conventional techniques such as neutralization, extraction, precipitation, chromatography, filtration and the like; or, alternatively, used in the next step without purification and/or isolation.

The synthesis of the compounds of the invention can be exemplified by, but is not limited to, the use of the intermediate 9.6 to prepare the compounds of the invention, as shown in Scheme 10.

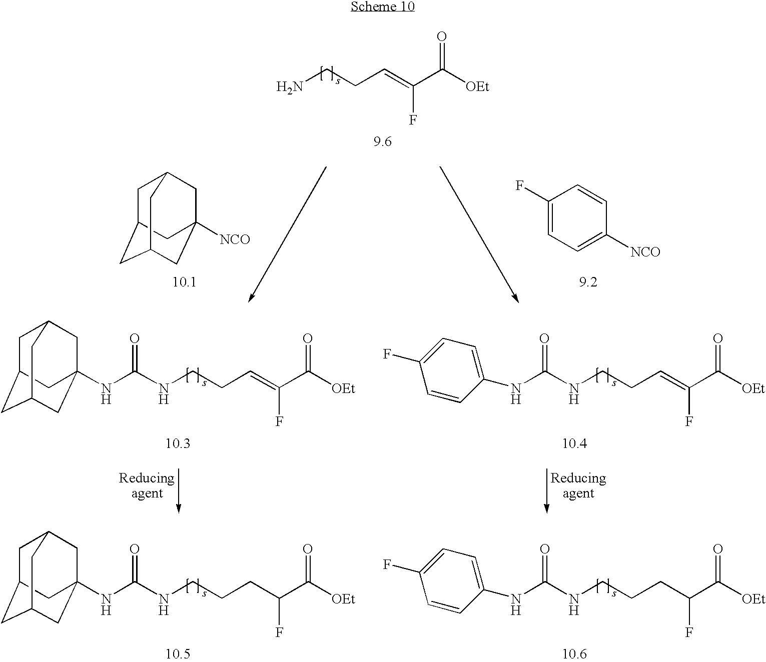

The intermediate 9.6 can be treated with appropriate isocyanate compounds 10.1 or 10.2 to form the corresponding adamantyl compounds 10.3 or phenyl compounds 10.4. Without limiting the scope of the present invention, Scheme 10 shows p-fluorophenyl or unsubstituted adamantyl for illustration purposes only. Any suitably substituted or unsubstituted phenyl or adamantyl can be used in Scheme 10 to yield the compounds of the invention. Typically, the reaction with isocyanates is conducted using DCM in the presence of triethylamine (TEA) at room temperature, or alternatively, a polar solvent such as DMF (dimethylformamide) at 0 to 10° C. Isocyanate compounds 10.1 or 10.2 can be either known compounds or compounds that can be prepared from known compounds by conventional synthetic procedures. Upon reaction completion, 10.3 and/or 10.4 can be recovered by conventional techniques such as neutralization, extraction, precipitation, chromatography, filtration and the like; or, alternatively, used in the next step without purification and/or isolation.

Compounds 10.3 or 10.4 can then be reduced using any suitable reducing agent known in the art, to give compounds 10.5 or 10.6, respectively. For example, 10.3 or 10.4 can be hydrogenated with palladium/carbon (Pd/C) in the presence of a suitable solvent known in the art such as, methanol, at suitable temperature such as, room temperature. Upon reaction completion, 10.5 and/or 10.6 can be recovered by conventional techniques such as neutralization, extraction, precipitation, chromatography, filtration and the like. Alternatively, the ester group of the adamantyl compounds 10.3 or phenyl compounds 10.4 can be hydrolyzed (not shown in scheme 10) to give the corresponding acid compounds. The hydrolysis of esters is well known in the art. For example, the ester can be hydrolyzed using lithium hydroxide (LiOH) in the presence of a suitable solvent such as, but not limited to THF/methanol/water. The resulting acids can then be reduced with reducing agents as described above to give the corresponding adamantyl or phenyl compounds of the invention.

A further elaboration of processes suitable for preparing compounds suitable for use in this invention are disclosed in McElroy et al, J. Med. Chem., 46:1066-1080 (2003); U.S. Pat. Nos. 6,693,130, 6,831,082, US Patent Application Publications 2007/0225283, 2006/0270609, 2008/0076770, 2008/0032978, 2008/153889, 2008/0207621, 2008/0207622, 2008/0200444, 2008/0200467, 2008/0207908, 2008/0227780, 2009/0023731, 2009/0082395, 2009/0082350, 2009/0082456, and 2009/0082423, U.S. Provisional Patent Application 61/046,316, filed on Apr. 18, 2008, and International applications WO2008/105968, WO2007/043652, WO2007/043653, WO2007/106705, WO2007/067836, WO2007/098352, WO2008/022171, WO2006/121719, WO2007/044491, WO2006/121684, WO2009/011872, WO2009/020960, and PCT/US2008/088244. All of which are herein incorporated by reference in their entirety.

The following examples are provided to illustrate certain aspects of the present invention and to aid those of skill in the art in practicing the invention. These examples are in no way to be considered to limit the scope of the invention.

### EXAMPLES

The examples below as well as throughout the application, the following abbreviations have the following meanings. If not defined, the terms have their generally accepted meanings.
aq.=aqueous
bd=broad doublet
bt=broad triplet
Boc=tert-Butoxycarbonyl
d=doublet
DCM=dichloromethane
DMAP=dimethylaminopyridine
DMF=dimethylformamide
DMSO=dimethylsulfoxide
eq.=equivalents
EtOAc=ethyl acetate
g=gram
HPLC=high performance liquid chromatography
LCMS=liquid chromatography mass spectroscopy
m=multiplet
M=molar
mg=milligram
MHz=megahertz
mL=milliliter
dL=deciliter
mM=millimolar
mmol=millimole
m.p.=melting point
MS=mass spectroscopy
N=normal
NMR=nuclear magnetic resonance
s=singlet
t=triplet
m=multiplet
TLC=thin layer chromatography
μL=microliter

### Example 1

### Compound Synthesis

### Synthesis of 1-Adamantyl-3-(1-(methylsulfonyl)piperidin-4-yl)urea (1)

A reactor was charged with 1.0 mole-equivalent of 4-piperidinecarboxamide, 16.4 mole-equivalents of THF, and 1.2 mole-equivalents of N,N-(diisopropyl)ethyl amine under a nitrogen atmosphere. The resulting mixture was cooled to 0-5° C. internal, and 1.2 mole-equivalents of methanesulfonyl chloride was added at such a rate as to maintain an internal temperature of less than 10° C. After addition was complete, the reaction mixture was stirred allowing the temperature to rise to 20° C. internal. The reaction contents was monitored until the amount of unreacted 4-piperidinecarboxamide was less than 1% relative to N-methanesulfonyl piperid-4-yl amide product (typically about 2-12 hours). The precipitated product was collected by filtration then washed with dichloromethane to remove excess (diisopropyl)ethylamine hydrochloride. The solid product was dried to constant weight in a vacuum oven under a nitrogen bleed maintaining an internal temperature of 50° C. to afford product as a light yellow solid in 87% yield. ¹H NMR (DMSO-d₆): 7.30 (s, 1H), 6.91 (s, 1H), 3.46-3.59 (m, 2H), 2.83 (s, 3H), 2.60-2.76 (m, 2H), 2.08-2.24 (m, 1H), 1.70-1.86 (m, 2H), 1.43-1.62 (m, 2H); MS: 207 [M+H]; m.p. 126-128° C.

A reactor was charged with 1.00 mole-equivalents of N-methanesulfonyl piperid-4-yl amide, 1.06 mole-equivalents of 1-adamantyl amine, and 39.3 mole-equivalents of acetonitrile, and the resulting mixture was heated to 40° C. internal under a nitrogen atmosphere. (Diacetoxyiodo)benzene (1.20 mole-equivalents) was charged portionwise in such a way that the reaction mixture was maintained below 75° C. internal. After the (diacetoxyiodo)benzene had been added, the reaction mixture was heated at 65-70° C. internal, and the reaction contents monitored until the amount of unreacted 1-adamantyl amine was less than 5% relative to product N-(1-methanesulfonyl piperidin-4-yl)-N′-(adamant-1-yl)urea (typically less than about 6 hours). The resulting mixture was cooled to 20° C. internal and filtered to remove a small amount of insoluble material. The filtrate was allowed to stand for 48 hours at which point the precipitated product was collected by filtration. The solid product was dried to constant weight in a vacuum oven under a nitrogen bleed maintaining an internal temperature of 50° C. to afford product in 58% yield based on N-methanesulfonyl piperid-4-yl amide. ¹H NMR (CDCl₃): 3.95-4.08 (m, 2H), 3.74-3.82 (m, 2H), 3.63-3.82 (m, 1H), 3.78 (s, 3H), 3.70-3.80 (m, 2H), 2.02-2.12 (m, 5H), 1.90 (s, 6H), 1.67 (s, 6H), 1.40-1.50 (m, 2H); MS: 356 [M+H]⁺; m.p. 228-229° C.

### Synthesis of 1-Adamantyl-3-(1-acetylpiperidin-4-yl)urea (3)

A reactor was charged with 1.00 mole-equivalent of 4-piperidinecarboxamide, 15.9 mole-equivalents of THF, and 1.23 mole-equivalents of N,N-(diisopropyl)ethylamine under a nitrogen atmosphere. The resulting mixture was cooled to 20° C. internal, and 1.10 mole-equivalents of acetic anhydride was added at such a rate as to maintain an internal temperature of less than 30° C. After addition was complete, the reaction mixture was stirred while maintaining an internal temperature of 20° C. The reaction contents was monitored until the amount of unreacted 4-piperidinecarboxamide was less than 1% relative to N-acetyl piperid-4-yl amide product (typically about 4-10 hours). The precipitated product was collected by filtration and washed with THF to remove excess (diisopropyl)ethylamine hydrochloride. The solid product was dried to constant weight in a vacuum oven under a nitrogen bleed while maintaining an internal temperature of 50° C. to afford the product as a white solid in 94% yield. ¹H NMR (CD₃OD): 4.48-4.58 (bd, 1H), 3.92-4.01 (bd, 1H), 3.08-3.22 (m, 1H), 2.62-2.74 (m, 1H), 2.44-2.53 (m, 1H), 2.12 (s, 3H), 1.88-1.93 (m, 2H), 1.45-1.72 (m, 2H); MS: 171 [M+H]; m.p. 172-174° C.

A reactor was charged with 1.00 mole-equivalents of N-acetyl piperid-4-yl amide, 0.87 mole-equivalents of 1-adamantyl amine, and 49.7 mole-equivalents of acetonitrile, and the resulting mixture was heated to 75° C. internal under a nitrogen atmosphere. (Diacetoxyiodo)benzene (1.00 mole-equivalents) was charged portionwise in such a way that the reaction mixture was maintained between 75-80° C. internal. After the (diacetoxyiodo)benzene was added, the reaction mixture was heated to 80° C. internal. The reaction contents was monitored until the amount of unreacted 1-adamantyl amine was less than 5% relative to product N-(1-acetylpiperidin-4-yl)-N′-(adamant-1-yl)urea (typically about 1-6 hours). After completion, the reaction mixture was cooled to 25° C. internal, and approximately 24 mole-equivalents of solvent was distilled out under vacuum while maintaining internal temperature below 40° C. The reaction mixture was cooled with agitation to 0-5° C. internal and stirred for an additional 2 hours. The technical product was collected by filtration and washed with acetonitrile. The crude product was dried to constant weight in a vacuum oven under a nitrogen bleed maintaining an internal temperature of 50° C. The dried, crude product was slurried with water maintaining an internal temperature of 20±5° C. internal for 4 hours and then collected by filtration. The filter cake was washed with heptane under a nitrogen atmosphere then dried to constant weight in a vacuum oven under a nitrogen bleed maintaining an internal temperature of 70° C. to afford product as a white solid in 72% yield based on 1-adamantyl amine. ¹H NMR (DMSO-d₆): 5.65-5.70 (bd, 1H), 5.41 (s, 1H), 4.02-4.10 (m, 1H), 3.61-3.70, (m, 1H), 3.46-3.58 (m, 1H), 3.04-3.23 (m, 1H), 2.70-2.78 (m, 1H), 1.98 (s, 3H), 1.84 (s, 6H), 1.64-1.82 (m, 2H), 1.59 (s, 6H), 1.13-1.25 (m, 1H), 1.00-1.12 (m, 1H); MS: 320 [M+H]⁺; m.p. 202-204° C.

### Synthesis of ethyl 2-fluoro-8-(3-adamantylureido)octanoate (4)

6-Amino-1-hexanol (9.00 g, 7.67 mmol) was taken in 300 mL of THF/Water (1:1) and to it was added tBoc anhydride (18.0 g, 8.44 mmol) followed by sodium carbonate (19.0 g, 19.2 mmol). The reaction mixture was then stirred at room temperature for 3 hours. After completion of the reaction, the resulting mixture was poured into water and extracted with ethyl acetate (2×300 mL). The combined organic layers were washed with water and brine and dried over sodium sulfate. Evaporation of the organic layer gave 16 g (96%) of tert-butyl 6-hydroxyhexylcarbamate which was essentially pure and was used without further purification.

tert-Butyl 6-hydroxyhexylcarbamate (16 g) was dissolved in 500 mL of DCM and to it was added 24.0 g of PCC and 60 g of neutral alumina. The reaction mixture was stirred at room temperature, and the progress of the reaction was monitored by TLC. The reaction was complete after 6 hours. The reaction mixture was filtered, and the filtrate was washed with water several times. The organic layer was evaporated under reduced pressure, and the crude product was purified by flash chromatography using ethyl acetate:hexane (1:3) as eluent to give tert-butyl 6-oxohexylcarbamate (14.4 g, 91%) as colourless oil.

tert-Butyl 6-oxohexylcarbamate (5.00 g, 2.74 mmol) was dissolved in 70 mL of dry THF and cooled to −78° C., and to it was added 12 mL of n-BuLi (1.6 M in hexane) and the solution stirred for 1 hour at −78° C. Triethyl-2-fluoro-2-phosphonoacetate (6.60 g, 2.74 mmol) dissolved in 20 mL of dry THF was added slowly to the reaction mixture via a cannula and the reaction mixture was allowed to warm to room temperature. The reaction mixture was then stirred at room temperature for 6 hours, poured into saturated ammonium chloride solution (200 mL), and extracted with ethyl acetate (2×300 mL). After evaporation of the organic layer, the crude product was purified by flash chromatography using ethyl acetate:hexane (1:4) as eluent to afford (Z)-ethyl 8-(tert-butoxycarbonylamino)-2-fluorooct-2-enoate (6.0 g, 68%).

(Z)-Ethyl 8-(tert-butoxycarbonylamino)-2-fluorooct-2-enoate (6.00 g, 1.78 mmol) was taken in 50 mL of DCM and to it was added 15 mL of TFA. The resulting mixture was stirred at room temperature for 2 hours. The reaction mixture was poured into water and extracted with DCM. The organic layer was washed with water and sodium bicarbonate solution, and, after drying over sodium sulfate, solvent was evaporated under reduced pressure. The crude product was purified by flash chromatography using ethyl acetate:hexane (2:3) as eluent to give (Z)-ethyl 8-amino-2-fluorooct-2-enoate (4.0 g, 95%). ¹H NMR (DMSO-d₆): δ 5.90-6.00 (m, 1H); 5.00 (bs, 2H); 4.20 (q, 2H); 3.20 (t, 2H); 2.60 (m, 2H); 1.60-1.80 (m, 6H); 1.40 (t, 3H). Mass: 204 (M+1, 100%).

(Z)-Ethyl 8-amino-2-fluorooct-2-enoate of Example 1 (2.0 g, 1.0 mmol) was dissolved in 50 mL of DCM and to it was added adamantyl isocyanate (1.7 g, 1.0 mmol) followed by triethylamine (2 mL, 2 mmol). The reaction mixture was stirred at room temperature for 6 hours. After completion of the reaction, the DCM layer was phase separated and washed with water several times. Evaporation of solvent gave the crude product which was purified by flash chromatography using ethyl acetate:hexane (2:3) as eluent to give (Z)-ethyl 2-fluoro-8-(3-adamantylureido)oct-2-enoate (3.4 g, 88%) as white solid. ¹H NMR (CDCl₃): δ 5.90-6.00 (m, 1H); 4.20 (q, 2H); 4.00 (bs, 2H); 3.20 (t, 2H); 2.60 (m, 2H); 2.00-1.80 (m, 6H); 1.70-1.40 (15H); 1.40 (t, 3H). Mass: 381 (M+1,100%).

(Z)-ethyl 2-fluoro-8-(3-adamantylureido)oct-2-enoate (2.0 g, 0.66 mmol) was taken in 20 mL of methanol and to it was added 350 mg of Pd/C (10%), and the reaction mixture was stirred at room temperature for 1.5 hours under a hydrogen atmosphere. After the reaction was complete, it was filtered through celite, the celite layer was washed with methanol, and the combined organic layers evaporated under reduced pressure. The crude product was purified by flash chromatography using ethyl acetate:hexane (2:3) as eluent to give ethyl 2-fluoro-8-(3-adamantylureido)octanoate (1.7 g, 93%) as a white solid. ¹H NMR (CDCl₃): δ 5.10-5.00 (m, 1H); 4.20 (q, 2H); 4.00 (bs, 2H); 3.20 (t, 2H); 2.60 (m, 2H); 2.00-1.80 (m, 7H); 1.70-1.40 (m, 15H); 1.40 (t, 3H). Mass: 383 (M+1,100%).

### Example 2

### Co-Administration of Compound 1 and EET Induced Relaxation of Rabbit Corpus Cavernosum

A study was performed using freshly isolated rabbit corpus cavernosum smooth muscle strips attached to a pressure transducer in a temperature controlled bath, to evaluate the efficacy of 1-adamantyl-3-(1-(methylsulfonyl)piperidin-4-yl)urea (Compound 1) for the treatment of erectile dysfunction.

The isolated rabbit corpus cavernosum strips were exposed to Compound 1 and/or 14,15-epoxyeicosatrienoic acid (14,15-EET) at 1 μM or 10 μM concentrations for 10 minutes. The relaxation of each strip was measured by pressure transduction. The percent relaxation was determined relative to a control strip incubated with sodium nitroprusside (SNP) of the formula Na₂[Fe(CN)₅NO].2H₂O, SNP is a compound that serves as a source of nitric oxide, a potent peripheral vasodilator. All experimental conditions were done in duplicate.

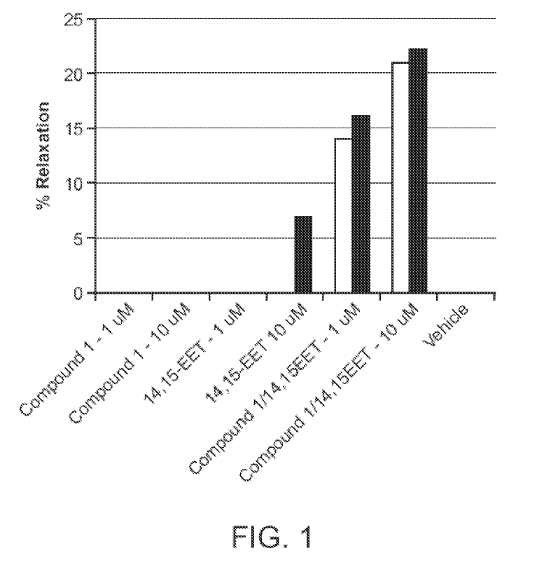

Compound 1 did not induce relaxation on its own, however, it enhanced the response to 14,15-EET markedly (FIG. 1). On its own, 14,15-EET had no effect at 1 μM but at 10 μM there was a 7% effect in one of the two experiments. At 1 μM of both Compound 1 and 14,15-EET, there was a 15% relaxation of the corpus cavernosum strip. The percent relaxation of the strip increased to 22% at 10 μM of both Compound 1 and 14,15-EET. Without being bound by theory, it is contemplated that the addition of Compound 1 prevents the hydrolysis of the exogenously provided 14,15-EET in addition to the endogenous EETs present in the corpus cavernosum to enhance the effects of EETs on relaxation of the strips.

These results demonstrate that the combination of both EET and the soluble epoxide hydrolase inhibitor supports a sEH mediated effect on rabbit corpus cavernosum smooth muscle relaxation, which is a crucial aspect of treating erectile dysfunction. The relatively modest amount of relaxation is not equivalent that seen with phosphodiesterase type 5 inhibitors. However, this is consistent with experiments by Yusif and Benter, supra, and suggests that the compounds of the invention are useful in treating erectile dysfunction. The compounds of the invention would be particularly useful in treating elderly and diabetic populations, which are contraindicated for treatment with phosphodiesterase type 5 inhibitors. Taken together, the above results indicate that, 1-adamantyl-3-(1-(methylsulfonyl)piperidin-4-yl)urea (Compound 1) is useful in treating erectile dysfunction. It is further contemplated that sEH inhibitors, for example compounds of any one of Formulas (A) and (I) to (V) and of Tables 1 and 2 are useful in treating erectile dysfunction.

### Example 3

### Effects of Compound 2 on Vascular Smooth Muscle Relaxation

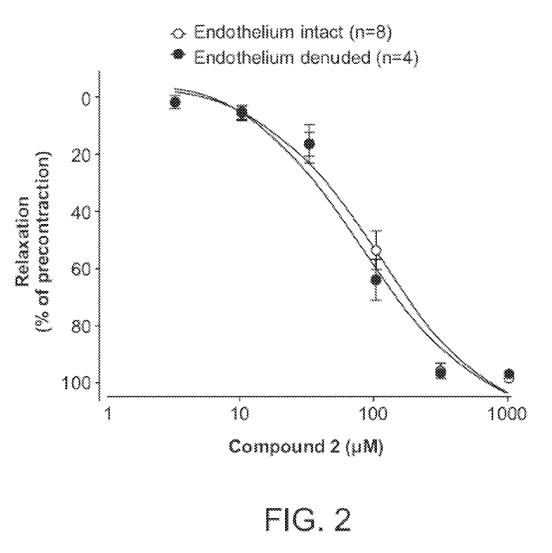

The efficacy of the 1-(1-nicotinoylpiperidin-4-yl)-3-(4-(trifluoromethoxy)phenyl)urea (Compound 2) to directly effect vasorelaxation of rat mesenteric arteries was examined. Rat mesenteric arteries with intact and denuded endothelium were isolated under microscope. Both intact and denuded arteries were exposed to various concentrations of Compound 2 (2 μM, 10 μM, 50 μM, 100 μM, 500 μM or 1000 μM) and the amount of relaxation was measured by a wired myograph. A dose dependent effect of Compound 2 was observed, i.e. as the amount of Compound 2 increased, the percent of relaxation increased (FIG. 2). For example, exposure to 10 μM of Compound 2 resulted in 5% relaxation, whereas exposure to 100 μM of Compound 2 resulted in 60% relaxation.

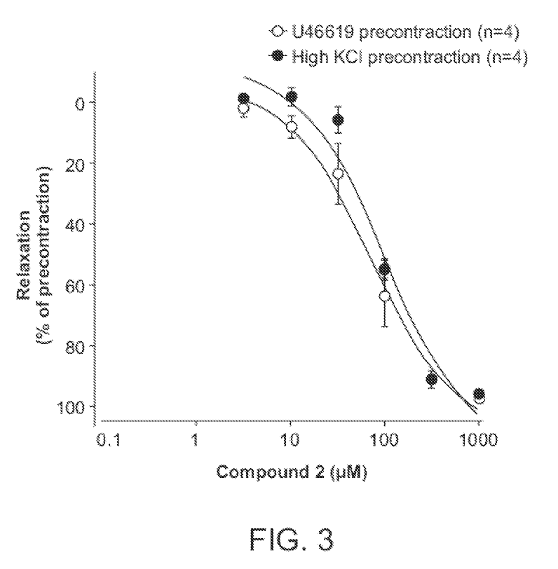

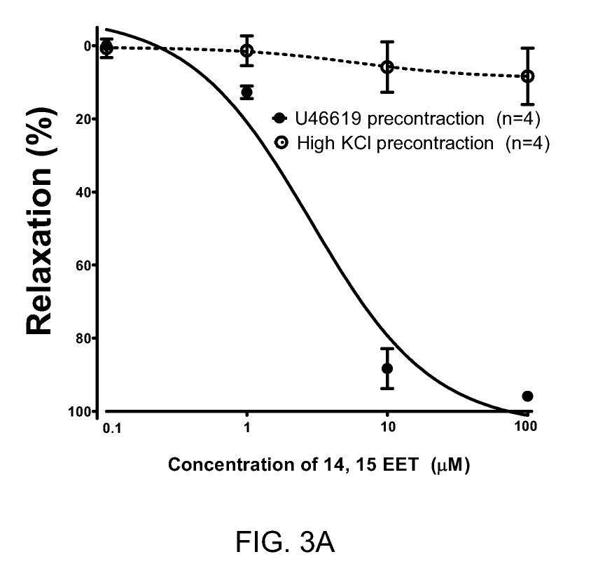

Additional experiments were performed wherein the pre-contraction of the mesenteric arteries was induced by pre-incubation with either 0.1-1.0 μM U46619 (9,11-dideoxy-9α,11α-methanoepoxy Prostaglandin F_{2α}, a thromboxane A2 agonist) or a high concentration of KCl (127 mM). The pre-contracted arteries were exposed to 2 μM, 10 μM, 50 μM, 100 μM, 500 μM or 1000 μM of Compound 2 and the level of relaxation was measured by myograph. Again a dose dependent effect of Compound 2 was observed (FIG. 3). The amount of relaxation induced by Compound 2 was consistent between the vessels pre-contracted by U46619 and high KCl. FIG. 3A shows the effect of 14,15-epoxyeicosatrienoic acid (EET) in rat mesenteric arteries pre-contracted with U46619 or high KCl under similar experimental conditions. Unlike the Compound 2, EET-induced vasorelaxation was completely blocked in the vessels precontracted with high KCl.

The above results indicate that 1-(1-nicotinoylpiperidin-4-yl)-3-(4-(trifluoromethoxy)phenyl)urea (Compound 2) is capable of relaxing the smooth muscle comprising the endothelium of rat mesenteric arteries. Furthermore, it is contemplated that the compounds of any one of Formulas (A) and (I) to (V) or of Tables 1 and 2 are capable of relaxing arteries.

### Example 4

### Rat Model of Vascular Smooth Muscle Relaxation

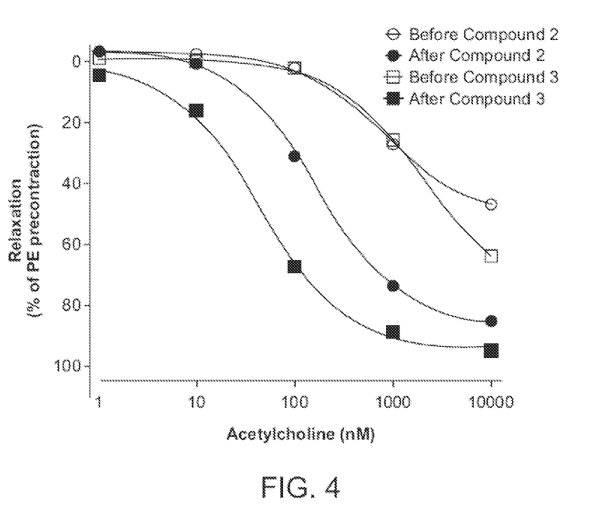

The efficacy of 1-(1-nicotinoylpiperidin-4-yl)-3-(4-(trifluoromethoxy)phenyl)urea (Compound 2) and 1-adamantyl-3-(1-acetylpiperidin-4-yl)urea (Compound 3) to effect vasorelaxation of rat arteries in vitro and in vivo was examined. Rats were infused with angiotensin II (ANG II) for 4 weeks resulting in a reduced vasorelaxation response to acetylcholine (Ach), a neurotransmitter known to cause endothelium-dependent vasodilation of arteries. Ach-induced relaxation was measured by myograph. In vitro pre-incubation of isolated rat arteries with either 100 μM of Compound 2 or 100 μM of Compound 3 for 90 minutes results in a significantly improved dose dependent response to Ach (FIG. 4). For example, with no exposure to either Compound 2 or Compound 3, the rat arteries showed 2% relaxation when incubated with 100 nM of Ach. However, pre-incubation with Compound 2 or Compound 3 resulted in 30% or 70% relaxation with 100 nM of Ach, respectively.

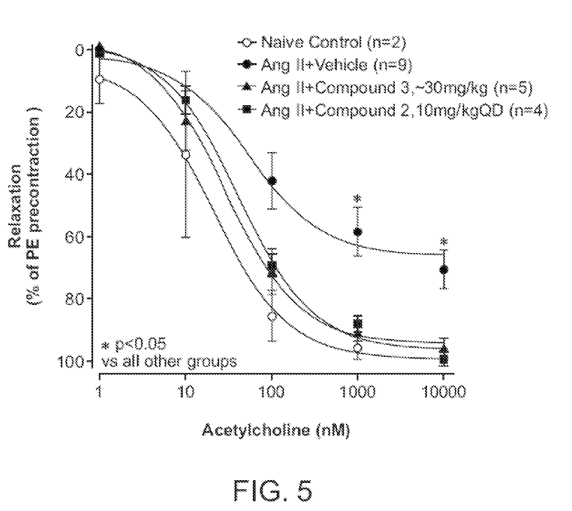

Additionally, in vivo attenuation of the effects of ANG II by Compound 2 or Compound 3 was examined. Rats were administered ANG II for 2 weeks and co-administered ANG II and either 10 mg of Compound 2 once a day by oral gavage or 30 mg of Compound 3 per day dose in drinking water for 2 weeks. Ach induced relaxation was measured by myograph. Similar to the in vitro experiments above, in vivo co-administration of Compound 2 or Compound 3 resulted in significantly improved dose dependent response to Ach (FIG. 5).

The above results indicate that the compounds 1-(1-nicotinoylpiperidin-4-yl)-3-(4-(trifluoromethoxy)phenyl)urea (Compound 2) and 1-adamantyl-3-(1-acetylpiperidin-4-yl)urea (Compound 3), are capable of improving the response to neurotransmitter induced relaxation of the smooth muscle comprising arteries both in vitro and in vivo. Furthermore, it is contemplated that the compounds of any one of Formulas (A) and (I) to (V) or of Tables 1 and 2 are capable of improving the response to neurotransmitter induced relaxation of the smooth muscle comprising arteries both in vitro and in vivo.

### Example 5

### Compound 3 Improves Vascular Smooth Muscle Response in Spontaneously Hypertensive Rats

The efficacy of 1-adamantyl-3-(1-acetylpiperidin-4-yl)urea (Compound 3) to improve the vasorelaxation response to Acetylcholine or Sodium Nitroprusside in spontaneously hypertensive rats was examined.

Spontaneously hypertensive rats (SHR) is an animal model of primary hypertension and is used to study cardiovascular disease. Hypertensive development begins around 5-6 weeks of age, reaching systolic pressures between 180 and 200 mmHg in the adult age phase. Starting between 40 and 50 weeks, SHR develop characteristics of cardiovascular disease, such as vascular and cardiac hypertrophy. Response to acetylcholine (Ach), which is a neurotransmitter known to cause endothelium-dependent vasodilation of arteries and sodium nitroprusside (SNP), which is a compound that serves as a source of nitric oxide, a potent peripheral vasodilator is significantly reduced in SHR (Yamamoto et al. J. Nutr. Sci. Vitaminol (Tokyo) 54(1):95-98 (2008)).

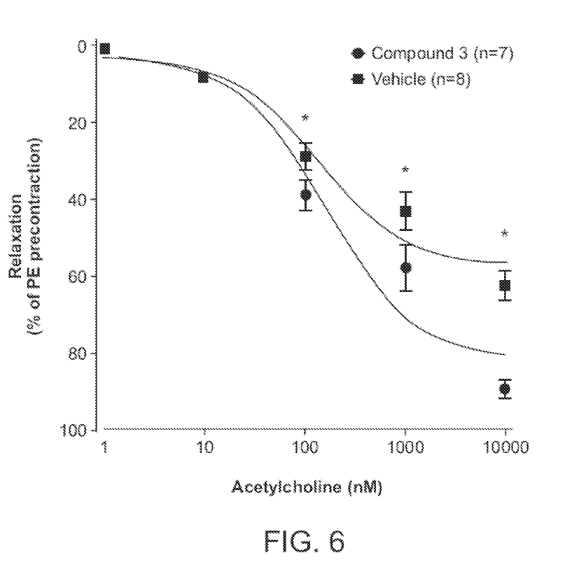

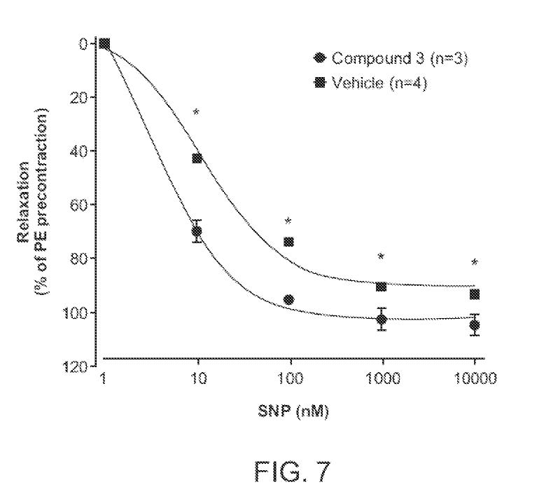

Aortic relaxation of isolated arteries from SHR was evaluated by myograph. A dose dependent response to Ach (FIG. 6) and SNP (FIG. 7) and a significantly improved response following pre-incubation with 100 μM of Compound 3 for 90 minutes were observed. For example, 10 μM of Ach induced relaxation of vehicle control (no compound) resulted in only 65% relaxation, whereas pre-incubation with Compound 3 improved relaxation of the artery to 90% (FIG. 6). As another example, 100 nM SNP induced relaxation of vehicle control (no compound) resulted in only 70% relaxation, whereas pre-incubation with Compound 3 improved relaxation to 95% (FIG. 7).

The above results indicate that 1-adamantyl-3-(1-acetylpiperidin-4-yl)urea (Compound 3), is capable of improving the response to neurotransmitter or nitric oxide induced relaxation of the smooth muscle comprising arteries of spontaneously hypertensive rats. Further it is contemplated that compounds of any one of Formulas (A) and (I) to (V) or of Tables 1 and 2 are capable of improving the response to neurotransmitter or nitric oxide induced relaxation of the smooth muscle comprising arteries of spontaneously hypertensive rats.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A method for enhancing non-vascular smooth muscle relaxation in a subject in need thereof, comprising administering to the subject an effective amount of a sEH inhibitor wherein said enhancement is unrelated to hypertension.

2. The method of claim 1, wherein the non-vascular smooth muscle comprises the reproductive tract of said subject.

3. The method of claim 1, wherein the non-vascular smooth muscle comprises the bladder of said subject.

4. The method of claim 1, wherein the non-vascular smooth muscle comprises the gastrointestinal tract of said subject.

5. The method of claim 1, wherein the sEH inhibitor is a compound of Formula (A):
R¹-L-C(=Q)NR²R^{2a} (A)
wherein:
L is selected from the group consisting of —NH—, —CR^{a}R^{b}—, —O—, a covalent bond, and —CR^{a}R^{b}NH—, where R^{a} and R^{b} are independently hydrogen or alkyl, or R^{a} and R^{b} together with the carbon bound thereto form a C₃-C₆ cycloalkyl;
Q is selected from the group consisting of O and S; and
R¹, R² and R^{2a} are independently selected from the group consisting of substituted alkyl, aryl, substituted aryl, heteroaryl, substituted heteroaryl, cycloalkyl, substituted cycloalkyl, heterocycloalkyl, and substituted heterocycloalkyl; or R² and R^{2a} together with the nitrogen bound thereto from an optionally substituted heterocycloalkyl;
provided that when neither R² nor R^{2a} is hydrogen, L is —NH—.

6. The method of claim 5, wherein the sEH inhibitor is a compound of Formula (I):
R¹NHC(=Q)NHR² (I)
wherein:
Q is selected from the group consisting of O and S; and
R¹ and R² are independently selected from the group consisting of substituted alkyl, aryl, substituted aryl, heteroaryl, substituted heteroaryl, cycloalkyl, substituted cycloalkyl, heterocycloalkyl, and substituted heterocycloalkyl; or
a stereoisomer, tautomer, or pharmaceutically acceptable salt thereof.

7. The method of claim 5, wherein R¹ is cycloalkyl, substituted cycloalkyl, phenyl or substituted phenyl.

8. The method of claim 5, wherein R² is substituted alkyl or substituted heterocycloalkyl.

9. The method of claim 5, wherein the sEH inhibitor is a compound of Formula (II) or (III): 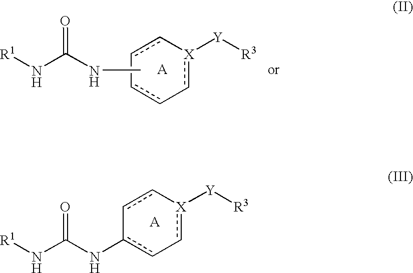
wherein:
R¹ is selected from the group consisting of aryl, substituted aryl, heteroaryl, substituted heteroaryl, cycloalkyl, substituted cycloalkyl, heterocycloalkyl, and substituted heterocycloalkyl;
X is CH, C or N; provided that when X is CH then ring A is cyclohexyl, when X is C then ring A is phenyl, and when X is N then ring A is piperidinyl;
Y is selected from the group consisting of CO, a covalent bond, O, and SO₂; and
R³ is selected from the group consisting of alkyl, substituted alkyl, heteroaryl, substituted heteroaryl, heterocycloalkyl and substituted heterocycloalkyl; or
a stereoisomer, tautomer, or pharmaceutically acceptable salt thereof.

10. The method of claim 5, wherein the sEH inhibitor is a compound of Formula (IV): 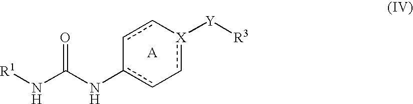
wherein:
R¹ is selected from the group consisting of aryl, substituted aryl, heteroaryl, substituted heteroaryl, cycloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂; and
R³ is selected from the group consisting of alkyl, substituted alkyl, heteroaryl, substituted heteroaryl, heterocycloalkyl and substituted heterocycloalkyl; or
a stereoisomer, tautomer, or pharmaceutically acceptable salt thereof.

11. The method of claim 10, wherein R¹ is adamantyl or substituted adamantyl.

12. The method of claim 10, wherein R¹ is phenyl or substituted phenyl.

13. The method of claim 5, wherein the compound is of Formula (V): 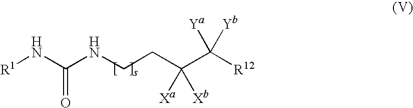
wherein
R¹ is selected from the group consisting of aryl, substituted aryl, heteroaryl, substituted heteroaryl, cycloalkyl, substituted cycloalkyl, heterocycloalkyl, and substituted heterocycloalkyl;
s is 0-10;
R¹² is selected from the group consisting of —CH₂OR¹³, —COR¹³, —COOR¹³, —CONR¹³R¹⁴, and carboxylic acid isostere; and
R¹³ and R¹⁴ are independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; or R¹³ and R¹⁴ together with the nitrogen atom bound thereto form a heterocycloalkyl ring having 3 to 9 ring atoms, and wherein said ring is optionally substituted with alkyl, substituted alkyl, heterocycloalkyl, oxo or carboxy; and
each of X^{a}, X^{b}, Y^{a}, and Y^{b} is independently selected from the group consisting of hydrogen, C₁-C₄ alkyl, substituted C₁-C₄ alkyl, and halo, provided that at least one of Y^{a} and Y^{b} is halo or C₁-C₄ alkyl; or
a stereoisomer, tautomer, or pharmaceutically acceptable salt thereof.

14. The method of claim 13, wherein R¹ is adamantyl or substituted adamantyl.

15. The method of claim 13, wherein R¹ is phenyl or substituted phenyl.

16. The method of claim 13, wherein at least one of Y^{a} and Y^{b} is halo.

17. The method of claim 5, wherein the compound is selected from the group consisting of
1-adamantyl-3-(1-(methylsulfonyl)piperidin-4-yl)urea,
1-(1-nicotinoylpiperidin-4-yl)-3-(4-(trifluoromethoxy)phenyl)urea,
1-adamantyl-3-(1-acetylpiperidin-4-yl)urea,
ethyl 2-fluoro-8-(3-adamantylureido)octanoate,
2-fluoro-8-(3-adamantylureido)octanoic acid,
1-cyclohexyl-3-(1-picolinoylpiperidin-4-yl)urea,
1-(1-(Isopropylsulfonyl)piperidin-4-yl)-3-(4-(trifluoromethyl)phenyl)urea,
4-(4-3-adamantan-1-yl-ureido)cyclohexyloxy)benzoic acid,
1-(1-Acetyl-piperidin-4-yl)-3-(3-trifluoromethyl-phenyl)-urea,
1-(1-(isopropylsulfonyl)piperidin-4-yl)-3-(4-(trifluoromethoxy)phenyl)urea,
1-(4-Methanesulfonyl-phenyl)-3-(4-trifluoromethyl-phenyl)-urea,
1-(1-(3,3-dimethylbutanoyl)piperidin-4-yl)-3-(4-(trifluoromethyl)phenyl)urea,
1-(1-Acetyl-piperidin-4-yl)-3-(4-trifluoromethyl-phenyl)-urea, and
1-(1-Methanesulfonyl-piperidin-4-yl)-3-(4-trifluoromethyl-phenyl)-urea,
or stereoisomer, tautomer, or a pharmaceutically acceptable salt thereof.

18. A method for enhancing vascular smooth muscle relaxation in a subject in need thereof, comprising administering to the subject an effective amount of a compound selected from the group consisting of
1-adamantyl-3-(1-(methylsulfonyl)piperidin-4-yl)urea;
1-(1-nicotinoylpiperidin-4-yl)-3-(4-(trifluoromethoxy)phenyl)urea; and
1-adamantyl-3-(1-acetylpiperidin-4-yl)urea.

19. A method for treating a non-vascular smooth muscle disorder in a subject in need thereof, wherein said smooth muscle disorder is characterized by an otherwise healthy smooth muscle which over or under responds to stimuli and is not hypertension, comprising administering to the subject an effective amount of a sEH inhibitor.

20. The method of claim 19, wherein the smooth muscle disorder is erectile dysfunction.

21. The method of claim 20, wherein the subject is unable to be treated by administration of an effective amount of a phosphodiesterase type 5 inhibitor.

22. The method of claim 20, wherein the subject is also suffering from a disorder selected from the group consisting of congestive heart failure, heart disease, stroke, hypotension and diabetes.

23. The method of claim 20, wherein the subject is over age 60.

24. The method of claim 19, wherein the smooth muscle disorder is uterine contractions.

25. The method of claim 19, wherein the smooth muscle disorder is overactive bladder.

26. The method of claim 19, wherein the smooth muscle disorder is irritable bowel syndrome.

27. The method of claim 19, wherein the sEH inhibitor is a compound of Formula (A):
R¹-L-C(=Q)NR²R^{2a} (A)
wherein:
L is selected from the group consisting of —NH—, —CR^{a}R^{b}—, —O—, a covalent bond, and —CR^{a}R^{b}NH—, where R^{a} and R^{b} are independently hydrogen or alkyl, or R^{a} and R^{b} together with the carbon bound thereto form a C₃-C₆ cycloalkyl;
Q is selected from the group consisting of O and S; and
R¹ and R² are independently selected from the group consisting of substituted alkyl, aryl, substituted aryl, heteroaryl, substituted heteroaryl, cycloalkyl, substituted cycloalkyl, heterocycloalkyl, and substituted heterocycloalkyl;
R^{2a} is selected from the group consisting of hydrogen, alkyl, substituted alkyl, aryl, substituted aryl, heteroaryl, substituted heteroaryl, cycloalkyl, substituted cycloalkyl, heterocycloalkyl, and substituted heterocycloalkyl;
or R² and R^{2a} together with the nitrogen bound thereto form an optionally substituted heterocycloalkyl;
provided that when L is not —NH—, R^{2a} is hydrogen.

28. The method of claim 27, wherein the sEH inhibitor is a compound of Formula (I):
R¹NHC(═O)NHR² (I)
wherein:
Q is selected from the group consisting of O and S; and
R¹ and R² are independently selected from the group consisting of substituted alkyl, aryl, substituted aryl, heteroaryl, substituted heteroaryl, cycloalkyl, substituted cycloalkyl, heterocycloalkyl, and substituted heterocycloalkyl; or
a stereoisomer, tautomer, or pharmaceutically acceptable salt thereof.

29. The method of claim 27, wherein R¹ is cycloalkyl, substituted cycloalkyl, phenyl or substituted phenyl.

30. The method of claim 28, wherein R² is substituted alkyl or substituted heterocycloalkyl.

31. The method of claim 28, wherein the sEH inhibitor is a compound of Formula (II) or (III): 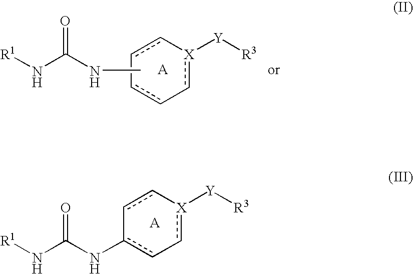
wherein:
R¹ is selected from the group consisting of aryl, substituted aryl, heteroaryl, substituted heteroaryl, cycloalkyl, substituted cycloalkyl, heterocycloalkyl, and substituted heterocycloalkyl;
X is CH, C or N; provided that when X is CH then ring A is cyclohexyl, when X is C then ring A is phenyl, and when X is N then ring A is piperidinyl;
Y is selected from the group consisting of CO, a covalent bond, O, and SO₂; and
R³ is selected from the group consisting of alkyl, substituted alkyl, heteroaryl, substituted heteroaryl, heterocycloalkyl or substituted heterocycloalkyl; or
a stereoisomer, tautomer, or pharmaceutically acceptable salt thereof.

32. The method of claim 31, wherein the sEH inhibitor is a compound of Formula (IV): 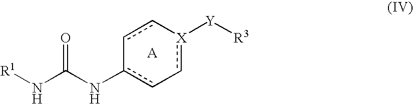
wherein:
R¹ is selected from the group consisting of aryl, substituted aryl, heteroaryl, substituted heteroaryl, cycloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂; and
R³ is selected from the group consisting of alkyl, substituted alkyl, heteroaryl, substituted heteroaryl, heterocycloalkyl or substituted heterocycloalkyl; or
a stereoisomer, tautomer, or pharmaceutically acceptable salt thereof.

33. The method of claim 32, wherein R¹ is adamantyl or substituted adamantyl.

34. The method of claim 32, wherein R¹ is phenyl or substituted phenyl.

35. The method of claim 28, wherein the compound is of Formula (V): 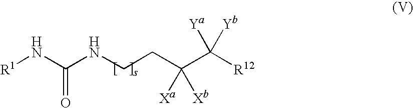
wherein:
R¹ is selected from the group consisting of aryl, substituted aryl, heteroaryl, substituted heteroaryl, cycloalkyl, substituted cycloalkyl, heterocycloalkyl, and substituted heterocycloalkyl;
s is 0-10;
R¹² is selected from the group consisting of —CH₂OR¹³, —COR¹³, —COOR¹³, —CONR¹³R¹⁴, or carboxylic acid isostere; and
R¹³ and R¹⁴ are independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; or R¹³ and R¹⁴ together with the nitrogen atom bound thereto form a heterocycloalkyl ring having 3 to 9 ring atoms, and wherein said ring is optionally substituted with alkyl, substituted alkyl, heterocycloalkyl, oxo or carboxy; and
each of X^{a}, X^{b}, Y^{a}, and Y^{b} is independently selected from the group consisting of hydrogen, C₁-C₄ alkyl, substituted C₁-C₄ alkyl, and halo, provided that at least one of Y^{a} and Y^{b} is halo or C₁-C₄ alkyl,
or a stereoisomer, tautomer, or pharmaceutically acceptable salt thereof.

36. The method of claim 35, wherein R¹ is adamantyl or substituted adamantyl.

37. The method of claim 35, wherein R¹ is phenyl or substituted phenyl.

38. The method of claim 35, wherein at least one of Y^{a} and Y^{b} is halo.

39. The method of any one of claims 19-26, wherein the compound is selected from the group consisting of:
1-adamantyl-3-(1-(methylsulfonyl)piperidin-4-yl)urea,
1-(1-nicotinoylpiperidin-4-yl)-3-(4-(trifluoromethoxy)phenyl)urea,
1-adamantyl-3-(1-acetylpiperidin-4-yl)urea,
ethyl 2-fluoro-8-(3-adamantylureido)octanoate,
2-fluoro-8-(3-adamantylureido)octanoic acid,
1-cyclohexyl-3-(1-picolinoylpiperidin-4-yl)urea,
1-(1-(isopropylsulfonyl)piperidin-4-yl)-3-(4-(trifluoromethyl)phenyl)urea,
4-(4-3-adamantan-1-yl-ureido)cyclohexyloxy)benzoic acid,
1-(1-acetyl-piperidin-4-yl)-3-(3-trifluoromethyl-phenyl)-urea,
1-(1-(isopropylsulfonyl)piperidin-4-yl)-3-(4-(trifluoromethoxy)phenyl)-urea,
1-(4-methanesulfonyl-phenyl)-3-(4-trifluoromethyl-phenyl)-urea,
1-(1-(3,3-dimethylbutanoyl)piperidin-4-yl)-3-(4-(trifluoromethyl)phenyl)urea,
1-(1-acetyl-piperidin-4-yl)-3-(4-trifluoromethyl-phenyl)-urea, and
1-(1-methanesulfonyl-piperidin-4-yl)-3-(4-trifluoromethyl-phenyl)-urea,
or a stereoisomer or a pharmaceutically acceptable salt of the compound of the stereoisomer.

40. A method for treating a vascular smooth muscle disorder in a subject in need thereof, wherein said vascular smooth muscle disorder is characterized by an otherwise healthy smooth muscle which over or under responds to stimuli and is not hypertension, which method comprises administering to the subject an effective amount of a compound selected from the group consisting of
1-adamantyl-3-(1-(methylsulfonyl)piperidin-4-yl)urea;
1-(1-nicotinoylpiperidin-4-yl)-3-(4-(trifluoromethoxy)phenyl)urea; and
1-adamantyl-3-(1-acetylpiperidin-4-yl)urea.
